(19) United States
(12) Patent Application Publication
(10) Pub. No.: US 2017/0224632 A1
(43) Pub. Date: Aug. 10, 2017

(54) Microparticle Enabled Delivery Structures, Methods of Preparing and Using Same

(71) Applicant: Bluegrass Advanced Materials, LLC, Lexington, KY (US)

(72) Inventors: Nihar Manilal Shah, Buffalo Grove, IL (US); James Hilt, Lexington, KY (US); Thomas Dziubla, Lexington, KY (US); Joe Wyse, Nicholasville, KY (US); Prachi Gupta, Jaipur (IN)

(21) Appl. No.: 15/347,910

(22) Filed: Nov. 10, 2016

Related U.S. Application Data
(60) Provisional application No. 62/253,827, filed on Nov. 11, 2015.

(51) Int. Cl. A61K 31/05; A61K 9/10; A61K 9/19; C08F 222/10; A61Q 19/08; A61Q 17/04; A61K 8/88; A61K 8/04; A61K 9/00; A61K 31/573

(52) U.S. Cl. CPC A61K 31/05; A61K 9/006; A61K 9/10; A61K 47/481; A61K 47/48215; A61K 9/19; A61K 31/573; A61Q 19/08; A61Q 17/04; A61K 8/88; A61K 8/04; C08F 222/1006; A61K 2800/522; C08F 2222/108; C08F 2222/1013; C08F 2222/1033

(57) ABSTRACT

The disclosed subject matter relates to the delivery of hydroxyl-containing compounds as microparticles for a variety of pharmaceutical, biomedical, cosmetics and personal care applications. This entails the manufacture and use of polymerized hydro-X compounds. Note is made of hydro-X compounds selected from the group consisting of curcuminoids, stilbenoids, resolvins, phenylethanoids, tocopherols, tocotrienols, flavanones, flavones, prenylflavonoids, isoflavones, isoflavanes, dihydrochalcones, isoflavenes, coumestans, lignans, flavonoligans, flavonols, mycoestrogens, xenoestrogens, phytoestrogens, sterols, corticosteroids, androgens, estrogens, stanols, steroids, secosteroids, tannins, statins, catechols, catechins, opioids, cannabinoids, pleuromutilins, luteolinidin, anthocyanidins, apigeninidin, glycosylated compounds, and macrolides.

### FIELD OF THE INVENTION

The disclosed subject matter relates to the delivery of hydroxyl-containing compounds as microparticles for a variety of pharmaceutical, biomedical, cosmetics and personal care applications. This entails the manufacture and use of polymerized hydro-X compounds. Note is made of hydro-X compounds selected from the group consisting of curcuminoids, stilbenoids, resolvins, phenylethanoids, tocopherols, tocotrienols, flavanones, flavones, prenylflavonoids, isoflavones, isoflavanes, dihydrochalcones, isoflavenes, coumestans, lignans, flavonoligans, flavonols, mycoestrogens, xenoestrogens, phytoestrogens, sterols, corticosteroids, androgens, estrogens, stanols, steroids, secosteroids, tannins, statins, catechols, catechins, opioids, cannabinoids, pleuromutilins, luteolinidin, anthocyanidins, apigeninidin, glycosylated compounds, and macrolides.

### BACKGROUND

Curcumin ((1E,6E)-1,7-Bis(4-hydroxy-3-methoxyphenyl)-1,6-heptadiene-3,5-dione) is a diarylheptanoid. Other curcuminoids are desmethoxycurcumin and bis-desmethoxycurcumin. Curcumin exists in several tautomeric forms, including a 1,3-diketo form and two equivalent enol forms. Resveratrol (3,5,4′-trihydroxy-trans-stilbene) is a stilbenoid, a type of natural phenol, and a phytoalexin. Resveratrol exists as two geometric isomers: cis-(Z) and trans-(E). The trans- and cis-resveratrol are either free or bound to glucose. The trans- form can undergo isomerization to the cis- form when exposed to ultraviolet irradiation. Hydrocortisone ((11β)-11,17,21-trihydroxypregn-4-ene-3,20-dione) is a steroid hormone belonging to the glucocorticoid class of hormones.

The aforementioned compounds present free hydroxyl (—OH) groups in their structures that enable their chemical modification, specifically, reversible chemical modification that allows retrieval of the original compounds without structural and/or chemical modification. For example, chemical groups are reversibly added to the hydroxyl groups by reaction with acyl halides or acid anhydrides. As described herein, reacting the hydroxyl-containing compounds with acryloyl chloride or acrylic anhydride attaches acrylate groups to the hydroxyl groups on these compounds via labile ester bonds to generate monomeric units of the compounds. On further reacting their acrylated forms with crosslinking molecules containing one or more primary or secondary amines via the Michael addition reaction yields poly(β-amino ester) polymers containing the compounds in the polymer backbone. Co-monomers containing one or more acrylate groups, such as poly(ethylene glycol) diacrylate, are also included during the polymerization reaction. These co-monomers serve a number of functions such as controlling the compound loading and altering hydrophobicity. The polymerization is typically carried out at about 50° C., but is also carried out at lower or higher temperatures. The polymers are synthesized in the form of linear or branched chains that are then dissolved or dispersed in appropriate solvents and converted into microparticles using various methods such as phase separation, precipitation, emulsification, solvent evaporation, spray drying, electrostatic spraying, precision particle fabrication. Alternatively, the polymers are synthesized as insoluble crosslinked networks, such as in containers as large chunks or in pans as films. These insoluble crosslinked polymers are then converted into microparticles using various micronization techniques such as cryogenic grinding, jet milling, ball milling, hammer milling, universal impact milling.

It is also possible to synthesize the polymer microparticles during the polymerization process itself. Like the aforementioned polymerization methods, in this method the compound acrylate, any acrylate co-monomer and the amine-based crosslinker are dissolved in a solvent and mixed to react. While the reagents are still reacting and in a liquid phase, microparticle formation is achieved by creating a microparticle emulsion of the reaction solution within an immiscible solvent serving as the continuous phase. A non-ionic surfactant (like Tween® 80 or Polysorbate 80) is used to stabilize the emulsion. One way to achieve formation of the microparticle emulsion is to immediately pour the reaction solution into an immiscible solvent containing a surfactant that is being homogenized with a high speed mixer/homogenizer. The shear forces generated by the high speed mixing break the reaction solution into microparticles resulting in a stable emulsion in the continuous phase. The stable emulsion obtained is then cured under stirring to complete the crosslinking process and ‘harden’ the particles. Another way to achieve formation of the microparticle emulsion is by using static mixers. In this case the reagents pre-dissolved separately in a solvent are passed through the first static mixer to quickly and thoroughly mix them together. Immediately thereafter, the mixed reaction solution is passed through another static mixer along with an immiscible solvent containing a surfactant to obtain a stable emulsion that is collected in a container. The stable emulsion obtained is then cured under stirring to complete the crosslinking process and ‘harden’ the particles.

Up on exposure to water, the ester bonds located within the polymer backbone are cleaved by hydrolysis to break down (degrade) the polymer and release the original compounds without any modifications. The polymer degradation happens over an extended period of time resulting in continuous sustained release of the original compound. One method to control the duration of degradation, and hence release of the compound, is by controlling the hydrophobicity of the polymer network. A way to achieve this is to use co-monomers of various hydrophilicities (e.g. more hydrophilic poly(ethylene glycol) diacrylate versus less hydrophilic 1,6-hexanediol diacrylate), and also to change their relative proportion in the polymer with respect to the acrylated compound. Similarly, crosslinker molecules of various hydrophilicities are also used to synthesize polymers with different degradation rates.

Reference is made to the following publications, the teachings of which are incorporated herein in their entirety, as are all publications cited herein.
Nanoparticle-and Microparticle-based Delivery Systems: Encapsulation, Protection and Release of Active Compounds, David Julian McClements, CRC Press; 1 edition (Aug. 12, 2014).
“Microspheres for Drug Delivery, Chapter 2 from BioMEMS and Biomedical Nanotechnology, Volume I.
Biological and Biomedical Nanotechnology, Mauro Ferrari, Abraham Lee, and James Lee, Springer, 2006.
Particle Size Measurements: Fundamentals, Practice, Quality, Merjus, H, Springer (2009).
Particle Size Measurements: Physics, Particle physics CTI reviews (2016).
Mucoadhesive Materials and Drug Delivery Systems 1st Edition, Vitaliy V. Khutoryanskiy (Wiley, 2014).

Advances in biotechnology, genomics, and combinatorial chemistry have led to the identification, purification and/or creation of a wide variety of new small molecule therapeutic compounds. Many of these compounds suffer from common problems such as low solubility, poor stability, systemic side effects, and/or poor bioavailability. As such, the means of drug delivery impacts the efficacy and potential for commercialization as much as the nature of the compound itself. Thus, there is a need for safer and more effective methods and devices for drug delivery.

Controlled release drug delivery systems are being developed to address many of the difficulties associated with traditional methods of administration. The most widely studied and implemented controlled release drug delivery systems employ devices such as polymer-based disks, rods, pellets, or microparticles to encapsulate the small molecule of interest and then release it at controlled rates for relatively long periods of time. Such systems offer several potential advantages over traditional methods of administration. First, drug release rates are tailored to the needs of a specific application; for example, providing a constant rate of delivery or pulsatile release. Second, controlled release systems provide protection of compounds that are otherwise rapidly destroyed in the human body. Finally, controlled release systems increase patient comfort and compliance by replacing frequent (e.g., multiple times daily) doses with infrequent (once per day or less) ones.

Biodegradable polymer microparticles are one of the most common types of drug delivery vehicles. Microparticles encapsulate many types of drugs including small molecules and are formulated for easy administration (e.g. using a syringe needle). Unfortunately, encapsulation-based microparticles technologies suffer from a number of critical limitations, the key ones being poor drug loading, difficulty of large-scale manufacturing, inactivation of drug during fabrication, and poor control of drug release rates. These disadvantages lead to poor bioavailability of the compound at the target disease site, thus jeopardizing the commercialization of an otherwise promising small molecule therapeutic compounds.

New microparticle delivery technologies are under development to overcome these limitations. One such new technology is described in Dziubla et al., U.S. Pat. No. 8,642,087. Disclosed are phenolic compounds converted into degradable poly(β-amino ester) polymers via their hydroxyl groups. Since the phenolic compounds of particular interest become part of the polymer backbone, very high drug loadings exceeding 20% of the polymer mass are achievable. These polymers degrade over long periods up on contact with water to slowly release the original compound in a sustained fashion.

### SUMMARY OF THE INVENTION

Disclosed herein is a polymerized hydro-X compound. In some embodiments the polymerized hydro-X compound is selected from the group consisting of curcuminoids, stilbenoids, resolvins, phenylethanoids, tocopherols, tocotrienols, flavanones, flavones, prenylflavonoids, isoflavones, isoflavanes, dihydrochalcones, isoflavenes, coumestans, lignans, flavonoligans, flavonols, mycoestrogens, xenoestrogens, phytoestrogens, sterols, corticosteroids, androgens, estrogens, stanols, steroids, secosteroids, tannins, statins, catechols, catechins, opioids, cannabinoids, pleuromutilins, luteolinidin, anthocyanidins, apigeninidin, glycosylated compounds, or macrolides. The polymer is, in some embodiments, in the form of a film, which may be milled or otherwise converted or prepared as microparticles.

Note is made of microparticles wherein at least about 90% of said particles are less than about 10 μm in diameter and about 50% are less than about 5 μm in diameter and further wherein at least about 90% of said particles are less than about 3 μm in diameter and about 50% are less than about 1 μm in diameter.

The film disclosed herein is, in some embodiments, configured such that the hydro-X compound is released from said polymer in a controlled steady state fashion with substantial release at (at least about 90% by weight of hydro-X compound) in from about 12 hours to about 3 days and further to about 4 weeks, with particular reference to at least about 1, 2, or 3 weeks. The same release profile is disclosed for microparticles of this invention.

In some embodiments the invention yet further includes a polymerized hydro-X compound in the form of a mucoadhesive suspension. Reference is made to such suspension where the hydro-X compound is curcumin or resveratrol. In specific embodiments said curcumin or resveratrol is in the form of microparticles.

Also disclosed is method of protecting the reactive chemical properties of hydro-X compound until substantial release by hydrolysis from poly(hydro-X) compound preparations by the steps of acrylating said hydro-X compound and reacting of said acrylated hydro-X with coreacting agents, including amine containing compounds yielding a degradable poly(hydro-X) polymer.

This invention further encompasses a method to treating a patient for osteoartritis, a chronic wound, or oral mucositis by the step of administering a therapeutically effective dose of a polymerized hydro-X compound with particular reference to a mucoadhesive solution of poly(curcumin) or poly(resveratrol) microparticles.

This disclosure includes a poly(curcumin) crosslinked film, with particular reference to said film is being a micronized preparation. In one embodiment the film is constituted as a poly(curcumin) mucoadhesive suspension.

This disclosure includes a poly(resveratrol) crosslinked film, with particular reference to said film is being a micronized preparation. In one embodiment the film is constituted as a poly(resveratrol) lotion or ointment.

This disclosure includes a poly(hydrocortisone) crosslinked film, with particular reference to said film is being a micronized preparation. In one embodiment the film is constituted as a poly(hydrocortisone) injectable liquid suspension.

### BRIEF DESCRIPTION OF THE DRAWINGS

FIG. 1. [C1] is a graph of three poly(curcumin) microparticle formulations, C60, C70 and C90, showing similar particle size distributions with mean particle diameters of 20.7±4.3, 20.8±2.7 and 22.7±0.28 μm respectively.

FIG. 2 [R3] is a graph of particle size distribution of R40 poly(resveratrol) microparticles with a mean particle diameter of 9.12±0.7 μm.

FIG. 3. [C2] is a graph of sustained curcumin release during hydrolytic degradation of various poly(curcumin) microparticle formulations in PBS (containing 0.1% w/w SDS) at 37° C. C60 poly(curcumin) microparticles degraded the fastest in about 12 hours, while C70 took 15 hours and C90 took 24 hours to completely hydrolyze. This trend shows that increasing amount of curcumin multiacrylate w.r.t. the co-monomer PEGDA makes the formulation more hydrophobic resulting in slower degradation and curcumin release.

FIG. 4 [C3] is an HPLC of degradation products of C70 poly(curcumin) microparticles after incubation for 26 hours in 0.1% SDS-PBS buffer at 37° C. Peaks eluting from the microparticle degradation sample coincide with those of pure curcumin standard at 7.3, 7.7 and 8.1 minutes respectively.

FIG. 5 [R1] is a graph of sustained resveratrol release during hydrolytic degradation of various poly(resveratrol) microparticle formulations in PBS (containing 0.1% w/w SDS) at 37° C. R20 poly(resveratrol) microparticles degraded the fastest in about 4 hours, while R80 took 8 hours completely degrade. This trend shows that increasing amount of resveratrol acetate w.r.t. PEGDA makes the formulation more hydrophobic resulting in slower hydrolysis and release.

FIG. 6 [C4] shows antioxidant activity profile of the release products from degradation of poly(curcumin) microparticles. The antioxidant activity follows sustained curcumin release over time (FIG. 4) confirming retention of curcumin's activity.

FIG. 7 [R2] shows the antioxidant activity profile of the release product from degradation of poly(resveratrol) microparticles. The antioxidant activity follows sustained resveratrol release over time (FIG. 4) confirming retention of resveratrol's activity.

FIG. 8 [C5] comprises images of pig buccal tissues taken after application of different poly(curcumin) microparticles followed by continuous saliva flow over the tissue. Digital images were taken at t=0 and t=6 hours in order to identify the best mucoadhesive formulation. C70 composition with RTAAP of 1.0 shows the most retention of the poly(curcumin) microparticles after 6 hours as compared to other microparticles compositions.

FIG. 9 [C6] charts protein oxidation or indirectly carbonyl content is considered to be a major marker of oxidative stress in tissues, cell lines etc. Controls NOC and NOT showed total protein content of 0.37±0.25 and 0.95±0.17 nM carbonyls/mg of protein. While the OM induced samples showed a quiet high amount of carbonyl levels of up to 3.89±0.013 nM carbonyl/mg of protein and OM induced with poly(curcumin) treated samples helped in bringing down the carbonyl content to 1.97±0.76 nM carbonyl/mg of protein clearly showing the protection from OM induced oxidative stress.

FIG. 10 [C7] is a graph of extended sustained curcumin release from poly(curcumin) microparticles, with duration dependent on curcumin mixed acrylate (“CMA”) mole % and co-monomer type. X=Degradation Completed; - - - -=Degradation Ongoing.

FIG. 11 [R5] plots conversion of free trans-resveratrol suspended in PBS into cis-resveratrol upon exposure to simulated sun-equivalent UV light at 365 nm.

FIG. 12 [R6A] plots the change in the fraction of trans-Resveratrol of the total resveratrol content after exposure to simulated UV Sunlight.

FIG. 13 [R6B] is a plot showing higher relative trans-resveratrol content released from poly(resveratrol) compared to free resveratrol at each UV exposure time point.

FIG. 14 [R7] is a plot of the antioxidant activity of R40 poly(resveratrol) microparticles suspended in PBS exposed to simulated UV light.

FIG. 15 is a plot of loss of fluorescence from oxidation of fluorescein by peroxide radical generated by AAPH. In the absence of any protective antioxidant (black x), the fluorescence reaches minimum in about 25 minutes. In the presence of free resveratrol at 10 μg/ml (black +), the decay in fluorescence is delayed to about 110 minutes, but no further protection is offered as the resveratrol has been consumed. In case of R80 microparticles (filled symbols), after 1 hour of insult the antioxidant activity begins to recover with time because of continued release of active resveratrol as the polymer degrades.

FIG. 16 is a plot of the HPLC chromatogram confirming the conversion of hydrocortisone into its acrylate form. Peak A=original hydrocortisone, Peak B=hydrocortisone monoacrylate, Peak C=hydrocortisone diacrylate.

FIG. 17 is a graph of sustained hydrocortisone release during hydrolytic degradation of H60 poly(hydrocortisone) microparticles in PBS (containing 0.1% w/w SDS) at 37° C.

FIG. 18 Is a schematic showing synthesis of crosslinked poly(curcumin) by reaction between CMA, acrylate co-monomer and primary diamine crosslinker. Hydrolytic cleavage of ester bonds releases the original curcumin (OH-A-OH) molecule.

FIG. 19 is a schematic showing synthesis of crosslinked poly(triamcinolone acetonide) by reaction between triamcinolone acetonide (TAA) diacrylate, acrylate co-monomer and primary diamine crosslinker. Hydrolytic cleavage of ester bonds releases the original TAA (OH-A-OH) molecule.

FIG. 20 is a synthesis schematic. RvD1 is PEGylated in FIG. 20 (top line) and incorporated into the crosslinked poly(curcumin) network in FIG. 20 (second and third lines) during reaction of CMA, acrylate co-monomer and di-primary amine crosslinker. Hydrolytic cleavage of ester bonds releases the original curcumin (OH-A-OH) and RvD1 molecules.

FIG. 21 is a schematic representation of preparation and usage of poly(curcumin) oral rinse product to treat OM.

FIG. 22 is a schematic showing synthesis of crosslinked poly(resveratrol) by reaction between resveratrol triacrylate, acrylate co-monomer and primary diamine crosslinker. Hydrolytic cleavage of ester bonds releases the original resveratrol molecule.

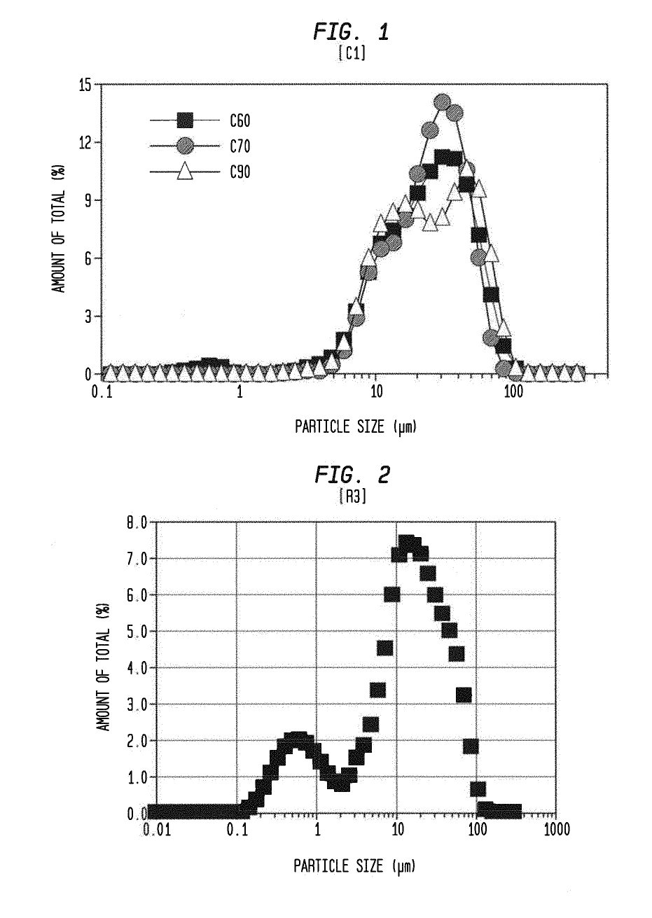

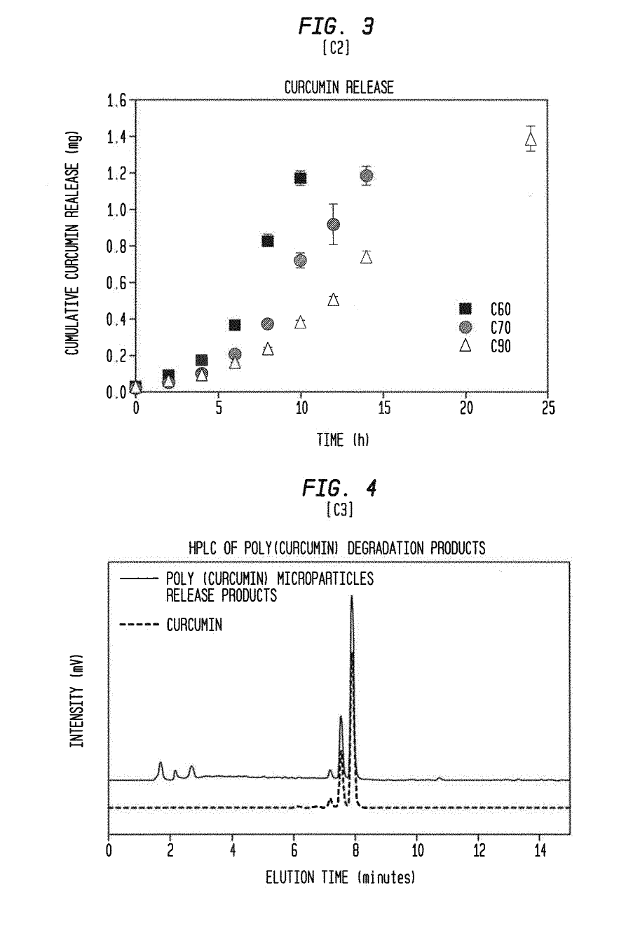

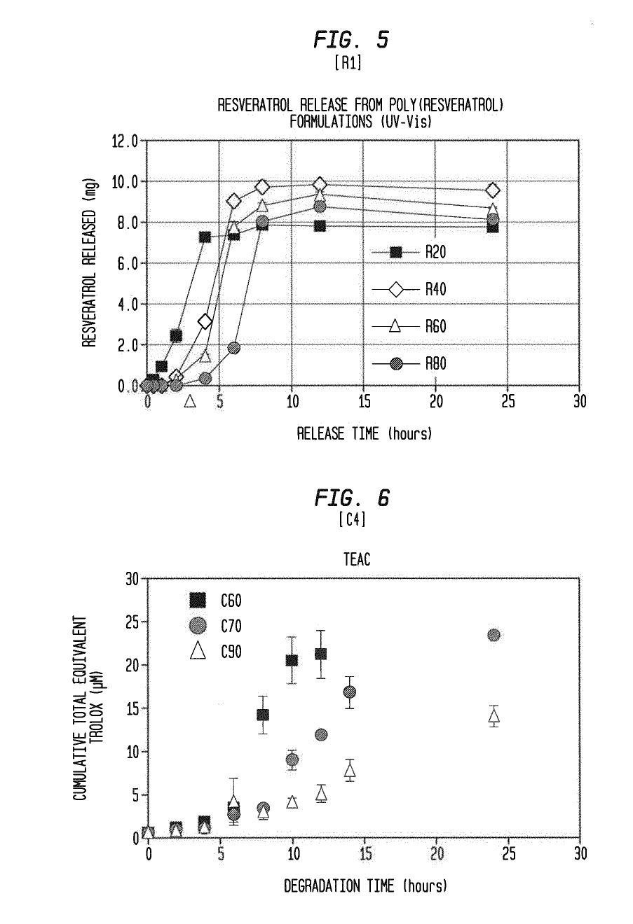

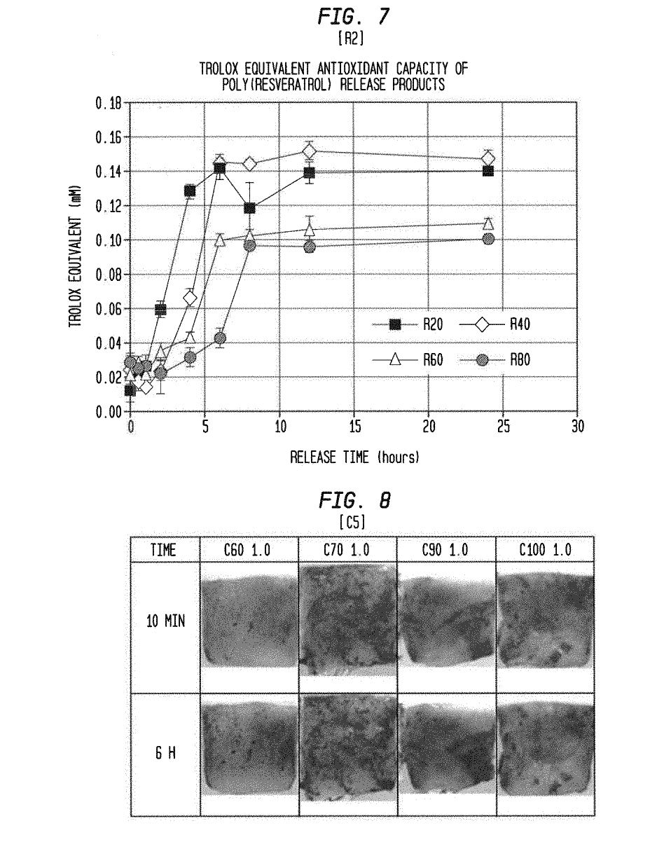

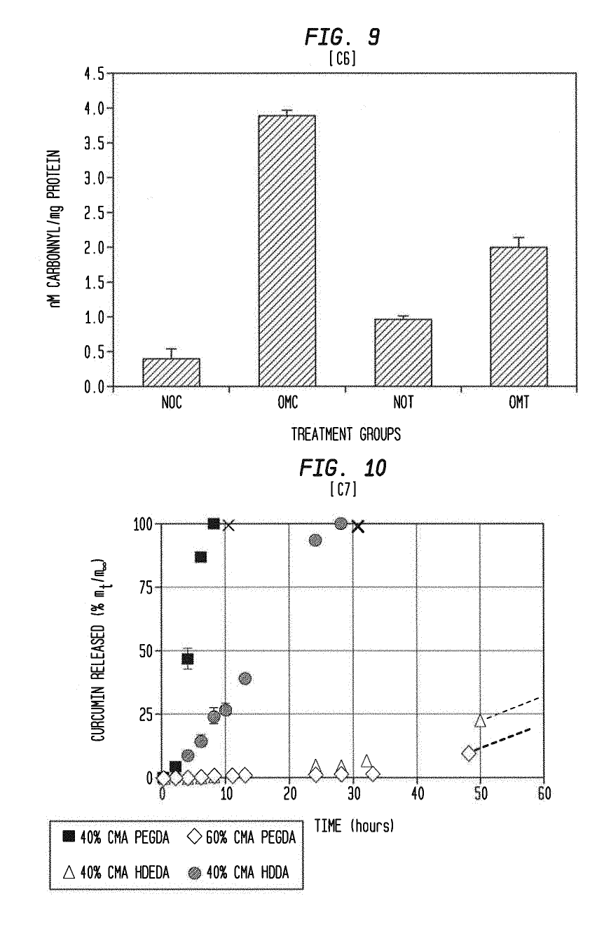

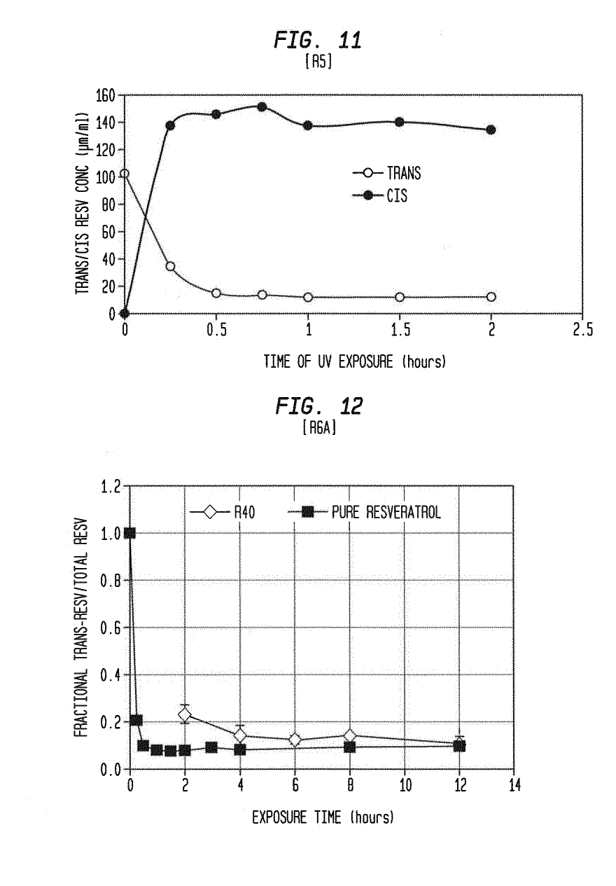

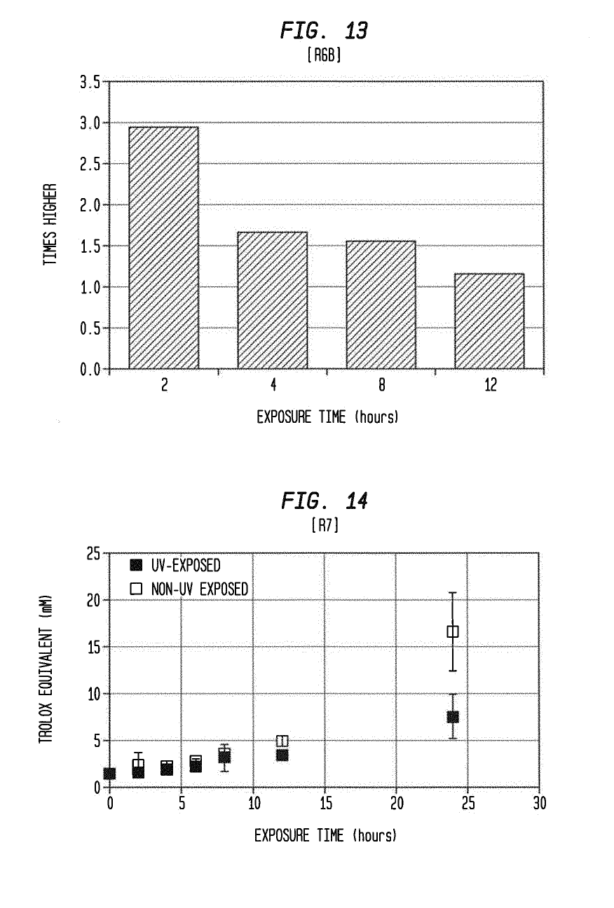

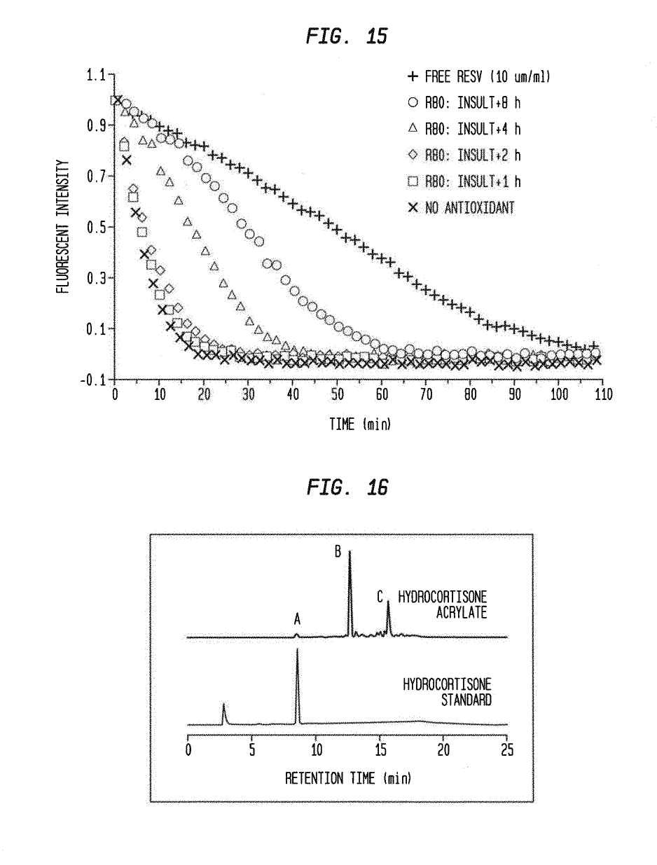

### DETAILED DESCRIPTION OF THE INVENTION

To more fully comprehend the invention, reference is made to the following definitions:

Micronized shall be understood to mean size particle diameters of less than about 40 μm, more preferably less than about 20 μm, yet more preferably less than about 15 μm, yet more preferably less than about 10 μm, yet more preferably less than about 5 μm and, in a particular embodiment less than about 4 μm, and more particularly preferably about 0.5-3 μm, with note of about 1-2 μm. Preferably, at least about 90% of said micronized composition has a particle size of less than about 10 μm and 5% less than about 5 μm as determined by the Malvern method. More preferably, 90% of the particles have a particle size of less than about 5 μm and 50% less than about 3 μm. By mean size is meant mass median diameter, determined by light scattering methods, for example using a Malvern Mastersizer® (Malvern, UK) or similar method.

The subject matter relates to the conversion of hydroxyl-containing compounds (as a group, “hydro-X” compounds) into degradable polymers which are processed into microparticles. Nonlimiting examples of this group includes curcumin, resveratrol, quercetin, ascorbic acid and hydrocortisone, and more broadly also include curcuminoids, stilbenoids, resolvins, phenylethanoids, tocopherols, tocotrienols, flavanones, flavones, prenylflavonoids, isoflavones, isoflavanes, dihydrochalcones, isoflavenes, coumestans, lignans, flavonoligans, flavonols, mycoestrogens, xenoestrogens, phytoestrogens, sterols, corticosteroids, androgens, estrogens, stanols, steroids, secosteroids, tannins, statins, catechols, catechins, opioids, cannabinoids, pleuromutilins, luteolinidin, anthocyanidins, apigeninidin, glycosylated compounds, and macrolides. Conversion of compounds into polymer microparticles enables controlled and sustained release of the original unmodified compound in a water-containing environment (e.g. the human body) due to the slow degradation of the polymer over time. The conversion of compounds into polymer microparticles also facilitates flexibility in physical formulation such as, but not limited to, dry powders, creams, ointments, gels, suspensions, lotions, films, tablets and capsules, as needed for a particular application and location of delivery in human body). Such formulations are used for a wide variety of applications including: viscous suspensions of curcumin polymer microparticles to treat oral mucositis, ointments containing curcumin polymer microparticles to treat dermal wounds, tablets containing corticosteroid polymer microparticles to treat gastrointestinal inflammatory diseases, lotions containing resveratrol polymer microparticles to treat skin ageing and wrinkling, injectable suspensions of corticosteroids to treat inflammatory joint diseases (e.g. osteoarthritis), capsules containing statin polymer microparticles to treat cardiovascular diseases, gels containing estrogen polymer microparticles to treat menopausal symptoms, oral films containing cannabinoid polymer microparticles to treat epilepsy.

The details of one or more embodiments of the presently-disclosed subject matter are set forth in this document. Modifications to embodiments described in this document, and other embodiments, is evident to those of ordinary skill in the art after a study of the information provided in this document. The information provided in this document, and particularly the specific details of the described exemplary embodiments, is provided primarily for clearness of understanding, and no unnecessary limitations are to be understood therefrom.

While the following terms are believed to be well understood by one of ordinary skill in the art, this invention is better understood with reference to the following definitions. Unless defined otherwise, all technical and scientific terms used herein have the same meaning as commonly understood by one of ordinary skill in the art to which the presently-disclosed subject matter belongs. Although many methods, devices, and materials similar or equivalent to those described herein can be used in the practice or testing of the presently-disclosed subject matter, representative methods, devices, and materials are now described. Following long-standing patent law convention, the terms “a”, “an”, and “the” refer to “one or more” when used in this application, including the claims. Thus, for example, reference to “a polymer” includes a plurality of such polymers, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations in some embodiments of 120%, in some embodiments of 110%, in some embodiments of 15%, in some embodiments of 11%, in some embodiments of 10.5%, and in some embodiments of 10.1% from the specified amount, as such variations are appropriate to perform the disclosed method. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The terms “active agent,” “bioactive agent,” “biologically active agent,” “therapeutic agent,” “pharmacologically active agent,” and “drug” are used interchangeably herein to refer to a chemical material or compound suitable for administration to a patient and that induces a desired effect. The terms include agents that are therapeutically effective as well as prophylactically effective. Also included are derivatives and analogs of those compounds or classes of compounds specifically mentioned that also induce the desired effect.

Microparticle (or micronized particle) shall mean a particle with an average diameter (i.e., the distance spanning the widest point, or points, of the microparticle) of about 0.1 μm to 200 μm. Microparticles may have regular or irregular shapes. Microparticle sizes are typically reported as an average diameter because microparticle formulations are typically composed of a population of particles with various diameters. Microparticles described with an average particle diameter typically have a particle size distribution such that they contain particles with diameters smaller than and larger than the reported average diameter.

Disclosed subject matter is based, at least in part, on the discovery that a modified non-free-radical polymerization technique, which makes use of the poly(β-amino ester) (PBAE) chemistry, provides a platform to synthesize PBAE polymers with various amounts and types of hydroxyl-containing compounds and various degradation properties.

In some embodiments, the biodegradable polymers of hydroxyl-containing compounds are configured to degrade over a time period and provide a sustained release of the original compound. In some embodiments of the presently-disclosed subject matter, an polymeric compound is provided that comprises a plurality of monomeric portions, where each monomeric portion includes a hydroxyl-containing compound molecule linked to one or more acrylate molecules, and where at least one acrylate molecule of each monomeric portion is linked by an amine crosslinker molecule to an acrylate molecule of an adjacent monomeric portion to thereby form a polymer.

The term “monomeric portion”, as used herein in reference to a portion of the presently-disclosed polymeric compounds is used to refer to a distinct unit or portion of the polymeric compound that comprises a hydroxyl-containing compound molecule linked to one or more acrylate molecules, and that then bonds with other molecules via the acrylate molecules to thereby form a polymeric compound of the presently-disclosed subject matter. In this regard, it is noted that the acrylate groups may act as functional groups that react and bond with other molecules, such that the monomeric portions are also referred to as functionalized compounds. It is further noted that the monomeric portions are, in some embodiments, comprised of a hydroxyl-containing compound molecule interposed between a first acrylate molecule connected to one portion of the compound and a second acrylate molecule connected to a second portion of the compound to thereby create an diacrylate compound or, in other words, a monomeric portion that includes two acrylate molecules. In other embodiments, a monomeric portion is comprised of a hydroxyl-containing compound molecule interposed between a first acrylate molecule that is connected to one portion of the compound, a second acrylate molecule that is connected to a second portion of the compound, and a third acrylate molecule that is connected to a third portion of the compound to thereby create a multiacrylate compound or, in other words, a monomeric portion that includes three or, in some embodiments, more than three acrylate molecules connected to the compound molecules.

The term “acrylic acid” or “acrylate” refers to chemical moieties having the formula: but which can be modified to include various groups including, but not limited to, methyl groups and salts. As such, the term “acrylic acid” or “acrylate” is further inclusive of methacrylic acid and acrylic acid salts (e.g., acryloyl chloride groups) which can be attached to a hydroxyl-containing compound molecule and then utilized as part of a monomeric portion of the presently-disclosed compounds. In some embodiments of the presently-disclosed subject matter, the acrylate molecules included in the monomeric portions of the polymeric compounds are selected from the group consisting of acrylic acid and methacrylic acid.

As is recognized by those of ordinary skill in the art, the linking of an hydroxyl-containing compound molecule to one or more acrylate molecules is accomplished by a variety of chemical and/or electrostatic bonds and depends on the particular compound molecule chosen for a particular polymeric compound or application. In some embodiments, an acid or alcohol spacer molecule having a variable length (e.g., lactone substitution, caprolactone) is utilized, which upon degradation releases a protected antioxidant that is then hydrolytically or enzymatically cleaved. In some embodiments, however, and as also indicated in the exemplary formulas of the diacrylate and multiacrylate compounds provided above, the acrylate molecules of each monomeric portion are linked to the compound molecules by an ester linkage that is formed via the reaction of a hydroxyl (—OH) group on the compound molecule with a reactive group on an acrylate molecule. For example, in some embodiments, compound having two hydroxyl groups is reacted with an acryloyl chloride molecule to produce a monomeric portion where the compound molecule is interposed between the two acrylate molecules via two ester linkages.

The term “hydroxyl-containing compound” includes, but not limited to, curcumin, resveratrol, quercetin, ascorbic acid and hydrocortisone. More broadly it also includes, but not limited to, a variety of classes of compounds such as curcuminoids, stilbenoids, resolvins, phenylethanoids, tocopherols, tocotrienols, flavanones, flavones, prenylflavonoids, isoflavones, isoflavanes, dihydrochalcones, isoflavenes, coumestans, lignans, flavonoligans, flavonols, mycoestrogens, xenoestrogens, phytoestrogens, sterols, corticosteroids, androgens, estrogens, stanols, steroids, secosteroids, tannins, statins, catechols, catechins, opioids, cannabinoids, pleuromutilins, luteolinidin, anthocyanidins, apigeninidin, glycosylated compounds, and macrolides. Any of these compounds have one or more hydroxyl (—OH) groups in their structures.

As noted above, each monomeric portion is linked by an amine crosslinker molecule to an acrylate molecule of an adjacent monomeric portion to thereby form the polymeric compounds of the presently-disclosed subject matter. The terms “amine”, “amine molecule,” “amine crosslinker,” and “amine crosslinker molecule” are used interchangeably herein to refer to molecules that comprise at least one primary amine or at least two secondary amines and that can be used to link together two monomeric portions of the presently-disclosed compounds. In embodiments where the amine molecule comprises one primary amine, the term “monoamine” can be used. In embodiments where the amine molecule comprises two amines, the term “diamine” can be used. In embodiments where the amine molecule comprises more than two amines, the term “multiamine” can be used. The term “amine” is used herein to refer to a functional group including a nitrogen atom with three single bonds to either hydrogen atoms or alkyl groups, with at least one alkyl group being required. Amines include primary amines, secondary amines, and tertiary amines. A primary amine is defined as a nitrogen atom bonded to two hydrogen atoms and one alkyl group (R—NH₂). A secondary amine is defined as nitrogen atom bonded to one hydrogen atom and two alkyl groups (R—NH—R). A tertiary amine is defined as a nitrogen atom bonded to three alkyl groups (R3N).

In some embodiments, the amine molecules included in compounds of the presently-disclosed subject matter are “primary diamine molecules,” which comprise primary amines, or “secondary diamine molecules,” which comprise secondary amines. In some embodiments, the amine molecules are selected from the group consisting of 4,7,10-trioxa-1,13-tridecanediamine (TTD), 2,2′-(ethylenedioxy)bis(ethylamine) (EDBE), and hexamethylenediamine (HMD), as well as biologically-derived diamines and multi-amines including, but not limited to, piperazine, spermine, spermidine, cadaverine, putrescine, and combinations of the foregoing.

With further regard to the amine molecules used in accordance with the presently-disclosed subject matter, in some embodiments, a polymeric compound is produced without the use of a diamine molecule. In some embodiments, a primary amine molecule, which is capable of attaching to two acrylate molecules of two separate monomeric portions is advantageously utilized to produce a polymeric composition of the presently-disclosed subject matter. In this regard, in some embodiments, the primary amine molecules is selected from the group consisting of isobutylamine (IBA) or n-butylmethylamine (BMA).

In some embodiments, a polymeric compound is synthesized where the polymer is in a “linear” configuration such that the polymer is not branched. In some embodiments of the presently-disclosed subject matter, a polymeric compound is synthesized where the polymer is in a “branched” configuration such that the polymer is not linear or crosslinked.

In some embodiments, an antioxidant polymeric compound is provided that is crosslinked. The term “crosslinked,” is used herein to refer to a polymer that does not have a linear or branched configuration, but instead has a configuration where the polymer chains are linked to one another by chemical bonds (e.g., covalent or ionic bonds), either between different polymer chains or different parts of the same polymer chain. For example, in some embodiments of the presently-disclosed subject matter, a primary diamine molecule (e.g., H₂N—R—NH₂) is included in the polymeric compound and is tetrafunctional such that each amine within the primary diamine molecule binds with up to two acrylate molecules included in the monomeric portions of the polymer to thereby create a crosslinked polymer. Of course, each amine of a diamine molecule of the presently-disclosed subject matter need not be bonded in every case to two acrylate molecules, but in some embodiments includes unreacted amine molecules that are linked to only one acrylate group of a monomeric portion or that include a bond to one or more hydrogen atoms in place of bonds to acrylate groups. In some embodiments, the amounts of unreacted amine molecules included in a polymeric compound is varied and configured for a particular application by varying the amounts of acrylates and amine molecules that are combined together to produce a polymeric compound of the presently-disclosed subject matter.

As noted above, the presently-disclosed closed subject matter is based, at least in part, on the discovery that poly(β-amino ester) (PBAE) chemistry is effectively used as a platform to synthesize PBAE polymers of hydroxyl-containing compounds with tunable properties, which may then advantageously be used to provide for the release of a desired amount of a compound for a particular application. In this regard, in some embodiments of the presently-disclosed compounds, the degradation rate of the polymeric compounds, or, in other words, the rate at which the polymeric compounds are broken down into smaller components to allow for the release of the compound, is controlled by selecting the types and amounts of the acrylate co-monomer, compound, and/or amine molecules in a particular polymeric compound of the presently-disclosed subject matter. For example, in some embodiments, a monomeric portion is combined with either TTD, EDBE, or HMD as it has been observed that polymeric compounds comprised of TTD diamine molecules are capable of degrading at a faster rate than compounds including EDBE or HMD diamine molecules. As another example, in some embodiments, one or more additional diacrylate molecules are incorporated into a polymeric compound, as described in further detail below, as it has been observed that compounds making use of certain diacrylates (e.g., poly(ethylene glycol) diacrylate) are capable of degrading at a faster rate than compounds making use of other diacrylate molecules (e.g., 1,6-hexanediol diacrylate).

In some embodiments of the presently-disclosed subject matter, the degradation rate of the polymer is varied by varying the ratio of total acrylate molecules or moieties to total amines within the compounds. The phrases “molar ratio of acrylate reactive groups to amine reactive groups”, “ratio of total acrylate to amine protons”, and “RTAAP”, are used herein to refer to the ratio of the number of acrylate reactive groups to amine protons in a mixture that react to form a polymeric compound of the presently-disclosed subject matter. For instance, a diacrylate has two acrylate reactive groups and a primary monoamine has two amine reactive groups (protons). Thus, a compound comprising one diacrylate and one primary monoamine has a RTAA of 1:1. Without wishing to be bound by any particular theory, it is believed that unreacted amines within a polymeric compound of the presently-disclosed subject matter accelerate the rate at which the polymeric compounds degrade by auto-catalyzing the degradation of the polymer. As such, in some embodiments, a polymeric compound is synthesized having a higher molar ratio of acrylate reactive groups to amine reactive groups such that the polymer is one that degrades at a slower rate due to the presence of fewer unreacted amines remaining in the polymeric compound. In some embodiments, a polymeric compound is synthesized having a lower molar ratio of acrylate reactive groups to amine reactive groups such that the polymer is one that degrades at a faster rate due to the presence of higher unreacted amines remaining in the polymeric compound. In some embodiments, the molar ratio of acrylate reactive groups to amine reactive groups range from about 0.2 to about 4.0 and any value in between.

As described herein above, in some embodiments, the degradation rate of the compounds of the presently-disclosed subject matter is increased by including one or more additional multi-acrylate molecules, herein referred to as “co-monomer”, which are not associated with the hydroxyl-containing compound molecule, in the presently-disclosed compounds such that the one or more co-monomer molecules are substituted for the hydroxyl compound containing monomeric portions and are linked to the amine molecules of the presently-disclosed polymeric compounds. In this regard, in some embodiments, the ratio of monomeric proportions to the one or more co-monomer molecules is about 0 percent, about 5 percent, about 10 percent, about 15 percent, about 20 percent, about 25 percent, about 30 percent, about 35 percent, about 40 percent, about 45 percent, about 50 percent, about 55 percent, about 60 percent, about 65 percent, about 70 percent, about 75 percent, about 80 percent, about 85 percent, about 90 percent, about 95 percent, about 100 percent. In some embodiments, the ratio of monomeric portions to the one or more co-monomer molecules is about 100 percent. In some embodiments, one or more co-monomer molecules included in the polymeric compounds are selected from, but not limited to, poly(ethylene glycol) diacrylate, diethylene glycol diacrylate, 1,3-butanediol diacrylate, 1,6-hexanediol diacrylate, and combinations thereof. Further, in some embodiments, which make use of poly(ethylene glycol) diacrylate as a diacrylate molecule to control the degradation rate of the compounds, a poly(ethylene glycol) diacrylate molecule having a particular molecular weight is selected for a particular application such that molecular weight of the poly(ethylene glycol) diacrylate molecule is used as a tunable parameter to control the degradation of the antioxidant polymeric compound.

Further provided, in some embodiments of the presently-disclosed subject matter, are methods for synthesizing a polymeric compound. By making use of the Michael addition reaction between a mono-, di- or multiacrylate molecule and an amine molecule, a method for synthesizing a polymeric compound is provided that does not involve radical polymerization, but yet is a capable of producing a polymeric compound with tunable properties.

In some embodiments, a method for synthesizing a polymeric compound is provided that includes combining an amount of acrylate molecules with an amount of amine molecules in a solution such that the acrylate molecules react with the amine molecules to thereby form a polymer. This reaction is performed with or without the application of heat. In some embodiments, heating the solution comprises heating the solution to a temperature of about 20° C., about 25° C., about 30° C., about 35° C., about 40° C., about 45° C., about 50° C., about 55° C., about 60° C., about 65° C., about 70° C., about 75° C., about 80° C., about 85° C., or about 90° C. In some embodiments, heating the solution comprises heating the solution to a temperature of about 40° C. to a temperature of about 85° C. In some embodiments, the reaction is performed at a temperature of about −20° C. to about 200° C. Of course, to produce the solution of acrylate molecules and amine molecules, any suitable solvent is used and is selected for a particular synthesis procedure as is recognized by those of ordinary skill in the art.

In some embodiments of the presently-disclosed methods, a polymer compound of the presently-disclosed subject matter is synthesized by further combining an amount of co-monomer molecules with an amount of amine molecules in a solution and adding an amount of a monomeric portion of the presently-disclosed subject matter, where the monomeric portion includes a hydroxyl-containing compound linked to one or more acrylate molecules, as described above. In this regard, and as also described above, the synthesis procedures are, of course, readily adapted to produce a particular polymeric compound having a desired amount of hydroxyl-containing compound or a desired degradation rate by controlling the amounts and types of the co-monomer molecules, acrylate molecules, compound molecules, and amine molecules that are combined together in the solution. In some embodiments, a linear or branched polymer is produced by the above-described methods. In some other embodiments, a crosslinked polymer is produced by the above-described methods.

The polymeric compounds are synthesized in the form of linear or branched chains are then dissolved or dispersed in appropriate solvents and converted into microparticles using various methods such as phase separation, precipitation, emulsification, solvent evaporation, spray drying, electrostatic spraying, precision particle fabrication, as is obvious to those skilled in the art.

Alternatively, the polymeric compounds are also synthesized in insoluble crosslinked form. For example, a reacting solution of an amount of monomeric molecules, co-monomer molecules and amine molecules is poured in a large cylindrical vessel to produce the crosslinked polymer as a single cylindrical piece. In another method, a reacting solution of an amount of monomeric molecules, co-monomer molecules and amine molecules is poured into a tray or pan with a large surface area to produce the polymer as thin films. The insoluble crosslinked polymers are then converted into microparticles using various micronization techniques such as cryogenic grinding, jet milling, ball milling, hammer milling, universal impact milling. For example, as is obvious to those skilled in the art, in one method the crosslinked polymers are cut or chopped into smaller pieces, and then micronized into microparticles using jet milling.

It is also possible to synthesize the polymer microparticles during the polymerization process itself. As described in the methods above, in this method the monomeric molecules, co-monomer molecules and the amine molecules are reacted together in a suitable solvent. While the molecules are still reacting and in a liquid phase, microparticle formation is achieved by creating a microparticle emulsion of the reaction solution within an immiscible solvent serving as the continuous phase. A non-ionic surfactant (like Tween 80 or Polysorbate) is used to stabilize the emulsion. On way to achieve formation of the microparticle emulsion is to immediately pour the reacting solution into an immiscible solvent containing a surfactant that is being homogenized with a high speed mixer/homogenizer. The shear forces generated by the high speed mixing break the reaction solution into microparticles resulting in a stable emulsion in the continuous phase. The stable emulsion obtained is then cured under stirring to complete the crosslinking process and ‘harden’ the particles. Another way to achieve formation of the microparticle emulsion is using static mixers. In case of static mixing the reagents pre-dissolved separately in a solvent are passed through the first static mixer to quickly and thoroughly mix them together. Immediately thereafter, the mixed reaction solution is passed through another static mixer along with an immiscible solvent containing a surfactant to obtain a stable emulsion that is collected in a container. The stable emulsion obtained is then cured under stirring to complete the crosslinking process and ‘harden’ the particles.

Lubricants and/or glidants are also added during or after microparticle formation, to prevent agglomeration of the microparticles. Some such lubricants and glidants include, but not limited to, talc, magnesium stearate, sodium stearate, calcium stearate, stearic acid, poly(ethylene glycol), sodium chloride, sodium lauryl sulfate, silicon dioxide, boric acid, sodium oleate, sodium acetate, sodium benzoate, corn starch, colloidal silica and DL-leucine.

For administration of a polymeric microparticles as disclosed herein, in some embodiments, administering an effective amount of a compound comprises applying the polymeric compound microparticles to a tissue or organ of a subject. In this regard, in some embodiments, the polymeric compound microparticles are administered topically to the organs and tissues of a subject as part of a cream or ointment formulation wherein the compounds are provided as an active ingredient in a carrier such as a cream base. As is recognized by those of ordinary skill in the art, various formulations for topical use include drops, tinctures, lotions, creams, solutions, and ointments containing the active ingredient and various supports and vehicles.

Various liquid and powder formulations are also prepared by conventional methods for inhalation into the lungs of the subject to be treated. For example, the polymeric compound microparticles are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. Capsules and cartridges of, for example, gelatin for use in an inhaler or insufflator may be formulated containing a powder mix of the desired polymeric compound microparticles and a suitable powder base such as lactose or starch. Additionally, the polymeric compound microparticles are suspended in a suitable liquid vehicle (e.g. water, ethanol) and delivered to tissues directly via injections. For example, suspensions of polymeric anti-inflammatory compound microparticles in water are injected into joints to treat arthritis, inflammation and pain.

Each of the formulations described herein, are also used in pulmonary delivery vehicles (dry powder inhalers, aerosols, multidose inhalers), buccal delivery systems (rapid release films, mucoadhesive films/patches), and/or as oral pharmaceutical excipients.

Regardless of the route of administration, the polymeric compounds of the presently-disclosed subject matter are typically administered in amount effective to achieve the desired response. The term “effective amount”, as used herein, refers to an amount of the polymeric compound sufficient to produce a measurable biological response (e.g., a reduction in oxidative stress). Actual dosage levels of the polymeric microparticles of the presently-disclosed subject matter are varied so as to administer an amount of the compound that is effective to achieve the desired response for a particular subject and/or application. The selected dosage level depends upon a variety of factors including the activity of the particular compound, formulation, the route of administration, combination with other drugs or treatments, severity of the condition being treated, and the physical condition and prior medical history of the subject being treated. Preferably, a minimal dose is administered, and dose is escalated in the absence of dose-limiting toxicity to a minimally effective amount. Determination and adjustment of a therapeutically effective dose, as well as evaluation of when and how to make such adjustments, are known to those of ordinary skill in the art.

In some embodiments of the presently-disclosed methods, the polymeric compounds administered to a subject are configured to degrade within the subject over a predetermined period of time so as to provide a sustained release of the compound molecule to the subject. In some embodiments, the polymeric compounds of the presently-disclosed subject matter are synthesized such that the compounds degrade in about 30 minutes to about 100 days. In some embodiments, the polymeric compounds degrade in about 30, 50, 60, 70, 80, 90, 100, 120, 140, 160, 180, 200, 220, 240, 260, 280, 300, 320, 340, or 360 minutes. In other embodiments, the compounds degrade in about 6, 7, 8, 9, 10, 12, 14, 16, 18, 20, 22, 24, 26, 28, 30, 32, 34, 36, 38, 40, 42, 44, 46, or 48 hours. In yet further embodiments, the compounds degrade in about 2, 2.5, 3, 3.5, 4, 4.5, 5, 6, 7, 8, 9, 10, 12, 14, 16, 18, 20, 25, 30, 35, 40, 45, 50, 55, 60, 65, 70, 75, 80, 85, 90, 95, or 100 days to thereby provide a sustained release of the compound.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including horses used for racing), poultry, and the like.

The presently-disclosed subject matter is further illustrated by the following specific but non-limiting examples.

### Example 1

### Poly(Curcumin) Film Synthesis

Poly(curcumin) crosslinked films were synthesized via a single-step Michael addition reaction between curcumin multiacrylate (CMA) and the primary diamine crosslinker, 4,7,10-Trioxatridecane-1,13-diamine (TTD). Poly(ethylene glycol) diacrylate (PEGDA, MW 575) was added as a diacrylate co-monomer along with CMA to control the degradation characteristics of the resulting films. Poly(curcumin) films with four different CMA:PEGDA mole ratios w.r.t. acrylate groups, 60:40, 70:30, 90:10 and 100:0 were synthesized. These films and subsequently their microparticles are abbreviated as C60, C70, C90 and C100 respectively. The amount of TTD required to synthesize the films was calculated based on three different ratios of total acrylate to amine protons (RTAAP) of 0.8, 1.0 and 1.2. Briefly, C60 poly(curcumin) film with a target mass of 2 g was synthesized by dissolving 0.913 g of CMA in 1.5 ml of anhydrous methyl ethyl ketone (MEK). PEGDA (0.735 g) and TTD (0.352 g) were reacted together for 5 minutes in 1.5 ml of MEK separately. After 5 minutes, the CMA solution was quickly added to the reacting PEGDA-TTD solution while gently vortexing the PEGDA-TTD solution at low rpm. This reacting solution was quickly poured into an aluminum dish, covered with foil and allowed to react for 1 hour at room temperature. After 1 hour, the aluminum dish was incubated at 50° C. for another 23 hours, after which the crosslinked poly(curcumin) film was peeled off from the dish for further processing. A similar procedure was followed to synthesize C70, C90 and C100 poly(curcumin) films with the three different RTAAP values.

Poly(Curcumin) Film Washing to Extract Leachables

Freshly synthesized poly(curcumin) films were washed in anhydrous acetone to leach out any unreacted monomer and uncrosslinked components. Each film was placed in a 50 ml centrifuge tube, filled with 20 ml of acetone and covered with foil. The sealed tube was rotated at 25-30 rpm for 4 hours with the acetone replaced every hour. Every hour, an aliquot of the acetone containing the leachables from each tube was stored at −20° C. for quantification of the lost curcumin using UV-Vis spectrophotometry. After washing, the films were lyophilized to remove residual solvent.

Micronization of Poly(Curcumin) Film

Lyophilized poly(curcumin) films were ground into microparticles under cryogenic conditions using a SPEX SamplePrep 6770 Freezer/Mill. Briefly, a 2 g film along with 1% w/w magnesium stearate (as a lubricant) was loaded into the polycarbonate vial assembly (SPEX Sample Prep). The loaded sample was pre-cooled under liquid nitrogen for 2 minutes followed by milling for 10 minutes with the stainless steel impactor moving at a speed of 15 cycles per second. After the milling cycle was complete, the vial assembly containing the poly(curcumin) microparticles was wrapped in paper towels and allowed to equilibrate to room temperature for about 1 hour. The microparticle sample was then retrieved and the mass noted. The microparticle samples were stored at −20° C. until further use.

Curcumin Particle Size Characterization

The particle size distribution of the poly(curcumin) microparticle samples was analyzed using a Shimadzu SALD-7101 UV particle size analyzer operated using WingSALD software (ver. 1.02, Shimadzu). The measuring cuvette was filled with DI water and used to blank calibrate the instrument. Next, about 2-3 mg of powder was added to the cuvette and mixed with the provided L-shaped stirrer and then sonicated for 2 minutes. The sample was measured in the instrument under stirring. The instrument software automatically provided the mean particle size and the size distribution for each sample. Each sample was measured in triplicate.

### Example 2

### Poly(Curcumin) Microparticle Degradation and Curcumin Release

Five milligrams of the poly(curcumin) microparticle samples from Example 1 was suspended in 10 ml of phosphate buffered saline (PBS, pH 7.4) by bath sonication for 2 minutes. Since curcumin has poor solubility in water, 0.1% w/w sodium dodecyl sulfate (SDS) was added to the PBS to ensure complete solubility of the released curcumin. The sample suspension was incubated at 37° C. in a water bath with shaking at 70 rpm. Every 2 hours, the sample was centrifuged at 5000 rpm for 5 minutes, and a supernatant replaced with fresh PBS. The supernatant was stored at −20° C. for further analysis. This step was repeated until the 24 hour time point or until the poly(curcumin) sample has completely degraded. The collected supernatants were analyzed using a Varian Cary 50 Bio UV-Vis spectrophotometer with the absorbance measured at 420 nm (peak absorbance wavelength of curcumin). A few of the supernatant samples were also analyzed using HPLC to verity the release of the original curcumin molecule by comparison with the chromatogram of curcumin standards.

Antioxidant Activity of Released Curcumin

The trolox equivalent antioxidant capacity (TEAC) assay was used to quantify the antioxidant activity of the curcumin released after hydrolytic degradation of the poly(curcumin) microparticle samples. The TEAC assay is a colorimetric assay used to determine the antioxidant capacity of samples based on the suppression of the absorbance of 2, 2′-azinobis-(3-ethylbenzothiazoline-6-sulfonate) (ABTS^{•+}) radical cations by antioxidants. Briefly, a 7 mM ABTS^{•+} radical solution was prepared by reacting equal volumes of solutions of ABTS (8 mg/ml) and potassium persulfate (1.32 mg/ml) in DI water for 16-20 hours. The ABTS^{•+} radical solution was diluted in PBS to obtain an absorbance not exceeding 0.4 at 734 nm for a 200 μl sample in a 96-well plate. This diluted solution was used as the working solution for the assay. To carry out the assay, 10 μl of trolox standard solutions (concentrations ranging from 0-0.225 mM) prepared in PBS and 10 μl of the degradation release samples were added to individual wells of a 96-well plate. To these wells, 200 μl of the ABTS^{•+} working solution was added and allowed to sit for 5 minutes in dark. After 5 minutes, absorbance was measured at 734 nm. Absorbance of the trolox standards were used to generate a standard calibration curve, which was then used to calculate the equivalent trolox concentration for the poly(curcumin) degradation supernatant samples.

### Example 3

### Tissue Adhesion Ability of Poly(Curcumin) Microparticle Formulations

Poly(curcumin) films with three CMA:PEGDA ratios (C60, C70 and C90 as described above) with each with three different RTAAP (0.8, 1.0 and 1.2) were synthesized and cryo-milled into microparticles following the exactly same procedure as described in Sections 2 and 3 above. These nine microparticle formulations were evaluated in vitro for their extent and duration of adhesive to porcine buccal tissue under simulated salivary flow.

Poly(Curcumin) Mucoadhesive Solution Preparation:

An oral barrier rinse that provides physical protection in addition to therapeutic effect of curcumin includes a viscous water-based mucoadhesive solution that serves as the physical barrier as well as the carrier for the poly(curcumin) microparticles. Mucoadhesive solution contains ingredients that adhere to a mucosal surface. Deionized water (100 ml) was stirred rapidly at 2000 rpm using an overhead mixer and a coil impeller. To this stirring water, 0.1 g of Noveon AA-1 Polycarbophil (Lubrizol, Wickliffe, Ohio) was added slowly in small batches over an hour. Once all the Noveon was added, the stirring speed was reduced to 1000 rpm and continued for at least 30 minutes to ensure complete dispersion and hydration of the Noveon. Thereafter the stirring speed was again increased to 2000 rpm. Next, 0.05 g of Carbomer 971 P NF (Lubrizol, Wickliffe, Ohio) was added to the water following the same procedure and stirred at 1200 rpm for 30 minutes to ensure complete dispersion. The stirring was again increased to 2000 rpm and 0.2 g of Eudragit L100 (Evonik, Parsipanny, N.J.) was added to the water following the same procedure and stirred at 1200 rpm for 30 minutes to ensure complete dispersion. Finally, the viscous mucoadhesive solution was partially neutralized by adding 18% w/w NaOH solution dropwise until the pH reached 5.65. The solution was further stirred at 1600 rpm for 10 min.

Artificial Saliva

A standardized formulation for artificial saliva for medical device and pharmaceutical testing has not been published by any regulatory body in the US. Therefore we adapted the formulation published by Marques et al. in 2011. Specifically, we prepared the SS1 formulation described in the publication. Briefly, potassium chloride (0.720 g), sodium chloride (0.600 g), potassium phosphate monobasic, (0.680 g), sodium phosphate dibasic, dodecahydrate (0.866 g), potassium bicarbonate (1.500 g), potassium thiocyanate (0.060 g) and citric acid (0.030 g) were dissolved in 95 ml of DI water. The pH was adjusted to 6.5 by adding few microliters of 37% HCl. This was designated as Solution A. Separately, a solution of calcium chloride dehydrate (0.220 g) was prepared in 50 ml of DI water. This was designated as solution B. Calcium chloride was not dissolved along with the other salts as it causes precipitation of other salts within 24 hours of storage.

Poly(Curcumin) Tissue Adhesion Test

A 50 ml polypropylene syringe was filled with artificial saliva, prepared by mixing 47.37 ml of solution A and 2.63 ml of solution B. The syringe was attached to a digital syringe pump (KDS210, KD Scientific, Holliston, Mass.) programmed to infuse at a flow rate of 0.5 ml/min. One end of a Tygon tubing (⅛″ ID) was attached to the syringe, while the other end was left open. The abattoir-derived porcine buccal tissues was laid flat and cut into a 1 inch×1 inch piece using a razor blade and dissection scissors. The tissue piece was then adhered to a 3 inch×2 inch glass slide using tissue adhesive (Gluture, Abbott Laboratories, Abbott Park, Ill.), such that one edge of the tissue was flush with one of short edges of the slide. Poly(curcumin) microparticle samples were prepared by thoroughly mixing 50 mg of microparticles in 1 g of mucoadhesive solution to give 1% w/w suspension. This suspension was immediately applied to the buccal tissue to coat the entire surface. After 60 seconds, the glass slide with the coated tissue was clamped vertically. A rectangular piece of filter paper, pre-wetted with artificial saliva, was placed on the glass slide such that the bottom edge of the paper overlapped with the top edge of the tissue. The open end of the Tygon tubing was then affixed on the filter paper, about 5 mmm above the top edge of the tissue. This set up allowed the artificial saliva to spread and flow across the entire surface of the buccal tissue. The syringe pump flow was started at 0.5 ml/min. Once a steady flow of saliva was established over the tissue within a few seconds, the flow rate was reduced to 100 μl/min for the rest of the experiment. Digital photographs of the tissues were taken at start of flow, and then after 10 min, 30 min, 60 min and then every hour until 6 hours.

TABLE 1
Table 1. A qualitative visual ranking of the tissue images
taken for all formulations at 6 hrs. Six independent reviewers,
blinded to the formulations, selected the three best images
and ranked them on visible tissue coverage by poly(curcumin)
microparticles. The 70% CMA poly(curcumin) formulation
with RTAAP 1.0 was selected most frequently and received
the highest ranking.

| | Selection | |
|---|---|---|
| Poly(curcumin)formulation | frequency | Rank sum |
| C70, RTAAP 1.0 | 6 | 14 |
| C60, RTAAP 0.8 | 5 | 13 |
| C70, RTAAP 0.8 | 4 | 7 |
| C90, RTAAP 1.0 | 1 | 2 |
| C60, RTAAP 1.0 | 0 | 0 |

### Example 4

### In Vivo Preclinical Study 1: Hamster Chemotherapeutic Model

Study 1 Protocol and Sampling

A total of 32 male golden Syrian hamsters (Harlan Laboratories, Indianapolis, Ind.) weighing 90 to 115 g were randomly divided into 4 groups as shown in Table 1. Animals in the control groups (1 and 2) remained disease free. Oral mucositis was induced in animals in the OM groups (3 and 4) by administration of 5-flurouracil (5-FU, 60 mg/kg) intraperitoneally on day 0 and 2, followed by abrasion of the left cheek pouch. Treatments were administered into the left cheek pouches using needle-less syringes once daily from day 0 until euthanasia, under mild isoflurane anesthesia. The control groups (1 and 3) received 200 μl of PBS while the treatment groups received 200 μl of 10% w/w suspension of poly(curcumin) microparticles (C70 PBAE formulation) in the mucoadhesive vehicle. C70-PBAE microparticle formulation was selected because this formulation scored highest in mucoadhesive amongst the three formulations analyzed for curcumin release, where C70 gave a uniform 12-15 hour curcumin release profile. On day 3, under ketamine (100 mg/kg) and xylazine (10 mg/kg) anesthesia, the left cheek pouches of the animals in the OM groups (3 and 4) were everted and mild erythema was created via dragging an 18 gauge needle in two 3 cm long parallel lines on the tissue surface. Once daily, all animals were weighed, their left cheek pouches were digitally photographed, and also visually scored for OM severity. Animals were given 0.1-0.2 mg/kg buprenorphine once or twice daily as needed. Animals with excessive inflammation of the cheek pouches or those under significant distress (indicated by drastically reduced food intake and activity) were euthanized before the end of the study. At the end of the study on day 11, all remaining animals were euthanized by CO₂ asphyxiation. The treated (left) and control (right) cheek pouches were then excised. Half of the total number of cheek pouches were flash frozen in liquid nitrogen and immediately stored at −80° C. for the tissue biomarker assays (TEAC and protein carbonyl, discussed later). The other half of the number of cheek pouches were processed for histological examination as described below.

TABLE 2
Design of the animal study and the treatment plan.

| | | | | 5-FU | Treatment |
|---|---|---|---|---|---|
| | | No. of | Injury | Injection | (200 μl), |
| No. | Group | Animals | Day | Days | Daily |
| 1 | No OM, | 5 | None | None | PBS |
| | Control (NOC) | | | | |
| 2 | No OM, | 5 | None | None | 10% w/w |
| | Treatment (NOT) | | | | poly(curcumin) |
| | | | | | suspension |
| 3 | OM, | 11 | 3 | 0 & 2 | PBS |
| | Control (OMC) | | | | |
| 4 | OM, | 11 | 3 | 0 & 2 | 10% w/w |
| | Treatment (OMT) | | | | poly(curcumin) |
| | | | | | suspension |

A statistically significant (p≦0.05, one-way ANOVA with Dunnett's test) reduction in OM severity was observed in the poly(curcumin) treated group versus water (sham) treated group. (Table 3)

TABLE 3

OM Score

| Animal | Vehicle | Poly(curcumin) |
|---|---|---|
| 1 | 1.5 | 0.5 |
| 2 | 4 | 0 |
| 3 | 5 | 1 |

Protein Carbonyl Content of Tissue Homogenate

Protein carbonyl content of the cheek tissues was quantified as a marker of protein oxidation, and therefore oxidative damage in the sample. The 2, 4-dinitrophenylhydrazine (DNPH) assay was used according the manufacturer protocol (Cayman Chemicals). DNPH on reaction with carbonyls forms the corresponding hydrazone, which are detected spectrophotometrically. Briefly, 200-500 mg of the tissues were homogenized in PBS (200 mg tissue per ml) followed by centrifugation at 3000 rpm for 10 min. 200 μl of the supernatant was mixed with 800 μl DNPH and incubated for 1 hour in dark at room temperature. An equal volume of supernatant was mixed with 800 μl of 2.5 M HCl instead of DNPH as the control group. After 1 hour, the protein was precipitated by adding 20% trichloroacetic acid (TCA) solution while incubating the samples in an ice bath. After 5 minutes, the samples were centrifuged and the supernatant discarded to remove excess DNPH. This precipitation step was repeated once more after which the protein pellets were re-suspended in 1:1 ethanol/ethyl acetate mixture. The samples were again centrifuged and the supernatant discarded. The protein pellets were re-suspended in guanidine hydrochloride and centrifuged once more. The supernatants were transferred into a 96-well plate (220 μl per sample per well) and absorbance was measured at 360 nm using a spectrophotometer. The protein carbonyl concentration (in nmol/ml) was calculated as follows:

$$Protein Carbonyl ( \frac{nmol}{ml} ) = ( \frac{A}{0.011} ) * 2.5$$

Where ‘A’ is the absorbance of the samples.

The protein carbonyl content was finally reported as nmol carbonyl per mg of total protein. Total protein content was quantified by treating the control (HCl) samples with guanidine hydrochloride solution in the ratio of 1:10 v/v, and then measuring the absorbance at 280 nm. Solutions of bovine serum albumin (BSA) were used as the standards to determine the total protein concentration. Reference is made to FIG. 6 shows antioxidant activity profile of curcumin released from degradation of poly(curcumin) microparticles.

### Example 5

### Extended Release Poly(Curcumin) Formulations

Additional poly(curcumin) films were synthesized as described above in section 1.1 using either 1,6-hexanediol diacrylate (HDDA) or 1,6-hexanediol ethoxylate diacrylate (HDEDA) as the diacrylate comonomers, and a mole ratio of 40:60 of CMA:HDDA or CMA:HDEDA w.r.t acrylate groups. The films were washed, dried and cryo-milled as described above. The poly(curcumin) microparticles made with HDDA or HDEDA were then degraded in PBS (pH 7.4 containing 0.1% SDS) and analyzed for curcumin release as described above.

### Example 6

### Poly(Resveratrol) Film Synthesis

Poly(resveratrol) crosslinked films were synthesized via a single-step Michael addition reaction between resveratrol acrylate (RA) and the primary diamine crosslinker, 4,7,10-Trioxatridecane-1,13-diamine (TTD). Poly (ethylene glycol) diacrylate (PEGDA, Avg. MW 575) was added as a diacrylate co-monomer along with RA to control the degradation characteristics of the resulting films. Poly(resveratrol) films with four different RA:PEGDA molar ratios, 20:80, 40:60, 60:40 and 80:20 were synthesized. These films and subsequently their microparticles a abbreviated as R20, R40, R60 and R80 respectively. The amount of TTD required to synthesize the films was calculated based on a ratio of 1.0 for the total acrylate to amine protons (RTAAP). Briefly, R40 poly(resveratrol) film was synthesized by dissolving 0.410 g of RA and 1.216 g of PEGDA in 1.660 ml of anhydrous dichloromethane (DCM). TTD (0.432 g) was separately dissolved in 1.660 ml of DCM. The TTD solution was quickly added to the RA+PEGDA solution while gently vortexing the PEGDA-TTD solution at low rpm. This reacting solution was quickly poured into an aluminum dish, covered with foil and incubated at 50° C. for another 24 hours. The crosslinked poly(resveratrol) film was peeled off the dish for further processing. A similar procedure was followed to synthesize R20, R60 and R80 poly(resveratrol) films.

Poly(Resveratrol) Film Washing to Extract Leachables

Freshly synthesized poly(resveratrol) films were washed in anhydrous acetone to leach out any unreacted monomer and uncrosslinked components. Each film was placed in a 50 ml centrifuge tube, filled with 20 ml of acetone and covered with foil. The sealed tube was rotated at 25-30 rpm for 4 hours with the acetone replaced every hour. Every hour, an aliquot of the acetone containing the leachables from each tube was stored at −20° C. for quantification of the lost resveratrol using UV-Vis spectrophotometry. After washing, the films were lyophilized to remove residual solvent.

Micronization of Poly(Resveratrol) Films:

The lyophilized poly(resveratrol) films were ground into microparticles under cryogenic conditions using a SPEX SamplePrep 6770 Freezer/Mill. Briefly, a 2 g film along with 3% w/w magnesium stearate (as a glidant) was loaded into the polycarbonate vial assembly (SPEX Sample Prep). The loaded sample was pre-cooled under liquid nitrogen for 2 minutes followed by milling for 10 minutes with the stainless steel impactor moving at a speed of 15 cycles per second. After the milling cycle was complete, the vial assembly containing the poly(resveratrol) microparticles was wrapped in paper towels and allowed to equilibrate to room temperature for about 1 hour. The microparticle sample was then retrieved and the mass noted. The microparticle samples were stored at −20° C. until further use.

Resveratrol Particle Size Characterization

R40 poly(resveratrol) film was synthesized as described in 1.1.1 above, but using anhydrous methyl ethyl ketone (MEK) as the solvent for polymerization. The film was then cryogenically ground into microparticles as described in 1.1.2 and 1.1.3 above. The particle size distribution of the R40 poly(resveratrol) microparticles was analyzed using a Shimadzu SALD-7101 UV particle size analyzer operated using WingSALD software (ver. 1.02, Shimadzu). The refractive index was set to 1.4. The measuring cuvette was filled with DI water and used to blank calibrate the instrument. Next, about 2-3 mg of powder was added to the cuvette and mixed with the provided L-shaped stirrer and then sonicated for 2 minutes. The sample was measured in the instrument under stirring. The instrument software automatically provided the mean particle size and the size distribution for each sample. Each sample was measured in triplicate.

### Example 7

### Poly(Resveratrol) Microparticle Degradation and Resveratrol Release

Poly(resveratrol) microparticles of R20, R40, R60 and R80 formulations described above were suspended in 1 L of phosphate buffered saline (PBS, pH 7.4) in a standard paddle-type USP dissolution apparatus. Each formulation was tested in triplicate. Since resveratrol has poor solubility in water, 0.01% w/w sodium dodecyl sulfate (SDS) was added to the PBS to ensure complete solubility of the released resveratrol. The concentration of the microparticles of each formulation per liter was adjusted so as to give a final resveratrol concentration of 10 mg/L based on the theoretical resveratrol loading in the respective formulation. The dissolution apparatus was kept at 37° C. with stirring at 100 rpm, following USP guidelines. At 0, 0.5, 1, 2, 4, 6, 8, 12 and 24 hours, a 1 ml aliquot was taken from each 1 L suspension, centrifuged at 5000 rpm for 5 minutes, and a supernatant stored at −20° C. for further analysis. The pellet was then re-suspended in 1 ml of fresh PBS containing 0.01% SDS and added back to the corresponding dissolution vessel. The collected supernatants were analyzed using a Varian Cary 50 Bio UV-Vis spectrophotometer with the absorbance measured at 305 nm (peak absorbance wavelength of resveratrol).

Antioxidant Activity of Released Resveratrol

The trolox equivalent antioxidant capacity (TEAC) assay was used to quantify the antioxidant activity of the resveratrol released after hydrolytic degradation of the poly(resveratrol) microparticle samples. The TEAC assay is a colorimetric assay used to determine the antioxidant capacity of samples based on the suppression of the absorbance of 2, 2′-azinobis-(3-ethylbenzothiazoline-6-sulfonate) (ABTS^{•+}) radical cations by antioxidants. Briefly, a 7 mM ABTS^{•+} radical solution was prepared by reacting equal volumes of solutions of ABTS (8 mg/ml) and potassium persulfate (1.32 mg/ml) in DI water for 16-20 hours. The ABTS^{•+} radical solution was diluted in PBS to obtain an absorbance not exceeding 0.4 at 734 nm for a 200 μl sample in a 96-well plate. This diluted solution was used as the working solution for the assay. To carry out the assay, 10 μl of trolox standard solutions (concentrations ranging from 0-0.225 mM) prepared in PBS and 10 μl of the degradation release samples were added to individual wells of a 96-well plate. To these wells, 200 μl of the ABTS^{•+} working solution was added and allowed to sit for 5 minutes in dark. After 5 minutes, absorbance was measured at 734 nm. Absorbance of the trolox standards were used to generate a standard calibration curve, which was then used to calculate the equivalent trolox concentration for the poly(resveratrol) degradation supernatant samples.

### Example 8

### Stability of Resveratrol Under Simulated Sunlight UV Exposure

Free resveratrol was dissolved in 20 ml of PBS (pH 7.4 with 0.1% SDS) at a concentration of 100 ug/ml. R40 poly(resveratrol) microparticles were suspended in 20 ml of PBS (pH 7.4 with 0.1% SDS) at an equivalent resveratrol dose concentration of 100 μg/ml. The samples were then poured into disposable 100 mm polystyrene petri dishes. The samples were then exposed to 365 nm UV light at 0.07 mW/cm², an intensity that is equivalent to UV exposure from natural sunlight on a non-cloudy summer afternoon. At 0, 2, 4, 6, 8, 12 and 24 hours, 1 ml aliquots were taken from each petri dish, centrifuged at 6000 rpm for 5 minutes, and the supernatants stored at −20° C. for further analysis. The pellets were then resuspended in 1 ml of fresh PBS (containing 0.1% SDS) and added back to their respective dishes. An additional 2 ml of deionized (DI) water was also added to each dish to compensate for water loss due to evaporation. The supernatant samples were then analyzed using HPLC to quantify the residual trans-resveratrol.

Antioxidant Activity of Free Resveratrol and Poly(Resveratrol) after UV Exposure

The UV exposed samples of free resveratrol and R40 poly(resveratrol) were then analyzed for their antioxidant capacity using the TEAC assay.

### Example 9

### Protection of Resveratrol from Free Radical Damage

Incorporating resveratrol triacrylate into poly(beta amino esters) (PBAE) hydrogel microparticles allows for the extended release of resveratrol over time and protection of resveratrol remaining in the particles from environmental insults. The sustained release of resveratrol from these systems could result in enhanced antioxidant performance over a longer timescale (relative to the free form of resveratrol) given that the released compound retains its antioxidant capacity.

In order to examine the protection provided by polymerization of resveratrol, a modified version of the oxygen radical absorbance capacity (ORAC) assay was conducted. Microparticles of R80 polymer were used for this assay, which were synthesized as described in Example 6.

Radical Insult

All experiments were performed with phosphate buffered saline (pH 7.40) containing 0.01 wt % tween-80 (PBS-T80). Stock solutions/suspensions of free resveratrol or R80 microparticles were made in buffer at a concentration 4 time greater than desired. A free radical insult was accomplished by adding 350 mM 2,2′-Azobis(2-amidinopropane) dihydrochloride (AAPH) at a 3:1 (v/v) ratio. After 1 hour of insult with AAPH, the free resveratrol and R80-MP systems were assayed with the ORAC assay and compared to resveratrol standards that received no insult. For the R80-MP system, the particles were centrifuged and washed before taking the sample after the 1 hour insult. Additionally, the R80 microparticles that had received the 1 hour AAPH insult were allowed to sit in buffer and samples were taken at t=1, 2, 4, and 8 hours post insult.

ORAC Assay

A 96-well plate was placed in an oven at 37° C. and filled with 200 uL of buffer and not used for assaying. For each experimental well, addition of 150 uL of the insult media or standard was followed by the addition of 30 μl of fresh 350 mM AAPH and 30 μl of 1.75 μM fluorescein. For consistent AAPH concentrations, resveratrol standards were made at concentrations 4 times higher than their desired value and diluted with 350 mM AAPH at a 3:1 (v/v) ratio immediately before adding to well plate. The fluorescence was monitored kinetically with measurements taken every minute using a fluorescent spectrometer with a microplate reader attachment.

### Example 10

### Synthesis of and Sustained Release from Poly(Hydrocortisone) Polymers

Functionalization of hydroxyl (—OH) groups of hydrocortisone (HCN) into acrylate esters was done by reacting hydrocortisone with acryloyl chloride. In one example, acryloyl chloride (6.72 ml, 82.77 millimoles) was added drop-wise to a solution of hydrocortisone (5 g, 13.79 moles) and trimethylamine (TEA, 6.72 ml, 82.77 millimoles) in anhydrous dimethylsulfoxide (DMSO), stirring continuously at 300 rpm. The reaction vessel was kept in a water bath during the addition of acryloyl chloride in order to dissipate heat generated from the exothermic reaction of acryloyl chloride with hydrocortisone. The reaction was allowed to proceed for about 2 hours in dark at room temperature under continuous purging with high purity nitrogen. The resultant product solution was then vacuum filtered to remove the TEA-HCl salt. A large excess (20×) of deionized water was then added to the hydrocortisone acrylate (HCNA) solution in DMSO to precipitate the acrylate product. The precipitated HCNA product was collected on filter papers by vacuum filtration. The filter papers were then soaked and stirred in ethyl acetate to dissolve the HCNA product. The HCNA solution in ethyl acetate was then washed with 2-3× excess volume of 0.1 M HCl solution in DI water followed by 0.1 M potassium carbonate solution in DI water to remove excess TEA and acryloyl chloride respectively. Residual water was removed by adding anhydrous magnesium sulfate to the ethyl acetate solution, which was then removed by vacuum filtration. The ethyl acetate was evaporated off using vacuum for 16-18 hours to obtain a dry, crystalline dark green HCNA powder. The HCNA product was stored at −20° C. until further use. The product was characterized using reverse-phase HPLC (Shimadzu Prominence) using a Phenomenex Luna 5 μm C18 column (4.6×250 mm) with water (with 0.1% w/w phosphoric acid) and acetonitrile as the mobile phase. The products (residual free HCN, monoacrylate and diacrylate) were detected at 245 nm using a Schimadzu prominence UV-Vis detector (SPD-20A) attached to the HPLC.

Poly(Hydrocortisone) Polymer Synthesis

Poly(hydrocortisone) crosslinked polymer was synthesized via a single-step Michael addition reaction between HCNA and the primary diamine crosslinker, 4,7,10-Trioxatridecane-1,13-diamine (TTD). 1,6-Hexanediol diacrylate (HDDA) was added as a diacrylate co-monomer along with HCNA to control the degradation characteristics of the resulting crosslinked polymers. Poly(hydrocortisone) polymer with a HCNA:HDDA molar ratio of 60:40 was synthesized. This polymer is abbreviated as H60. The amount of TTD required to synthesize the polymer was calculated based on a ratio of 1.0 between the total acrylate groups to amine protons (RTAAP). Briefly, H60 poly(hydrocortisone) polymer was synthesized by dissolving 0.200 g of HCNA and 0.058 g of HDDA in 263 μl of anhydrous methyl ethyl ketone (MEK). TTD (0.081 g) was separately dissolved in 263 μl of MEK. The TTD solution was quickly added to the gently vortexing HCNA+HDDA solution. This reacting solution was incubated at 50° C. for 24 hours.

Poly(Hydrocortisone) Polymer Washing to Extract Leachables

The freshly synthesized crosslinked poly(hydrocortisone) polymer was washed in anhydrous acetone to leach out any unreacted monomer and uncrosslinked components. The polymer piece was placed in a 5 ml centrifuge tube, filled with 4 ml of acetone and covered with foil. The sealed tube was rotated at 25-30 rpm for 4 hours with the acetone replaced every hour. Every hour, an aliquot of the acetone containing the leachables from each tube was stored at −20° C. for quantification of the lost hydrocortisone using UV-Vis spectrophotometry. After washing, the polymer was lyophilized to remove residual solvent.

Poly(Hydrocortisone) Polymer Degradation and HCN Release

Poly(hydrocortisone) polymer samples were suspended in phosphate buffered saline (PBS, pH 7.4) at a polymer concentration of 100 μg/ml, which corresponded to a theoretical HCN loading of 49 μg/ml. Since HCN has poor solubility in water, 0.1% w/w sodium dodecyl sulfate (SDS) was added to the PBS to ensure complete solubility of the released HCN. The sample suspensions were incubated at 37° C. in a rocking incubator with shaking at 25 rpm. At 0, 2, 4, 6, 22, 24, 26, 28, 30 and 46 hours a 1 ml aliquot was drawn and stored at −20 C until further analysis. The sample volume from each tube was replaced with fresh PBS at every time point. The collected samples were analyzed using a Varian Cary 50 Bio UV-Vis spectrophotometer with the absorbance measured at 245 nm (peak absorbance wavelength of hydrocortisone).

### Example 11

### Treatment of Osteoarthritis

The more frequently affected joints in osteoarthritis (OA) are the hands, knees, hips, and spine. Although articular cartilage destruction is the hallmark of OA, the whole joint is affected including the synovial lining, the underlying bone, and supporting connective tissue elements. Although the specific causes of OA are unknown, it is believed to be a combination of mechanical and molecular events in the affected joint. The involvement of inflammation in the disease progression, marked by symptoms such as joint pain, swelling and stiffness, is now well recognized. Specifically, the increased secretion of pro-inflammatory mediators, such as cytokines (e.g. IL-16), tissue necrosis factor-α, and reactive oxygen species in the joint triggers increased expression of multiple catabolic pathways such as cyclooxygenase-2, matrix metalloproteinases, and a disintegrin and metalloproteinase with thrombospondin-1 domains (ADAMTS)-4 and 5. These mechanisms culminate in the destruction of the cartilage, ligaments and underlying bone, leading to functional loss.

Lacking a medical cure, OA requires a multi-faceted management approach involving both non-pharmaceutical (patient education, physical therapy, weight reduction, dietary changes, etc.) and pharmaceutical (topical and oral analgesics and non-steroidal anti-inflammatory drugs (NSAIDs)) interventions. Unfortunately, the long-term oral use of analgesics such as acetaminophen is linked with renal failure, and of NSAIDs (viz. Naproxen, Celebrex) can cause serious cardiovascular (viz. myocardial infarction, stroke) and gastrointestinal (viz., peptic ulcers, perforations, and bleeds) side effects. These complications, along with the significant patient population intolerant/unresponsive to these drugs have made local intra-articular injections of hyaluronic acid (HA) (e.g. Synvisc) and corticosteroids (triamcinolone acetonide family) mainstays of OA treatment. Although intra-articular HA injections provide symptomatic relief (pain and functional) for 4-26 weeks, the onset of action is slow and the reported effects are modest. Moreover, considerable controversy exists regarding its efficacy, cost-effectiveness, and benefit-to-risk ratio. In addition, each HA treatment course requires multiple, weekly injections to be effective. Meanwhile, intra-articular corticosteroids have proven effective in controlling pain for only 1-4 weeks, due to the short intra-articular resident times (3-6 days for the most commonly used intra-articular corticosteroids, triamcinolone acetonide and triamcinolone hexacetonide).

More importantly, no disease-modifying drugs are currently available to slow the progression of OA. However, animal studies have indicated that intra-articular injections of corticosteroids at much lower doses (sufficient to suppress catabolism) can have a disease-modifying effect by normalizing cartilage proteoglycan synthesis and significantly reducing the incidence and severity of cartilage erosion and osteophytes. This finding suggests that an approach is needed to improve the IA bioavailability of corticosteroids and thus enhancing their symptom-relieving and disease-modifying ability.

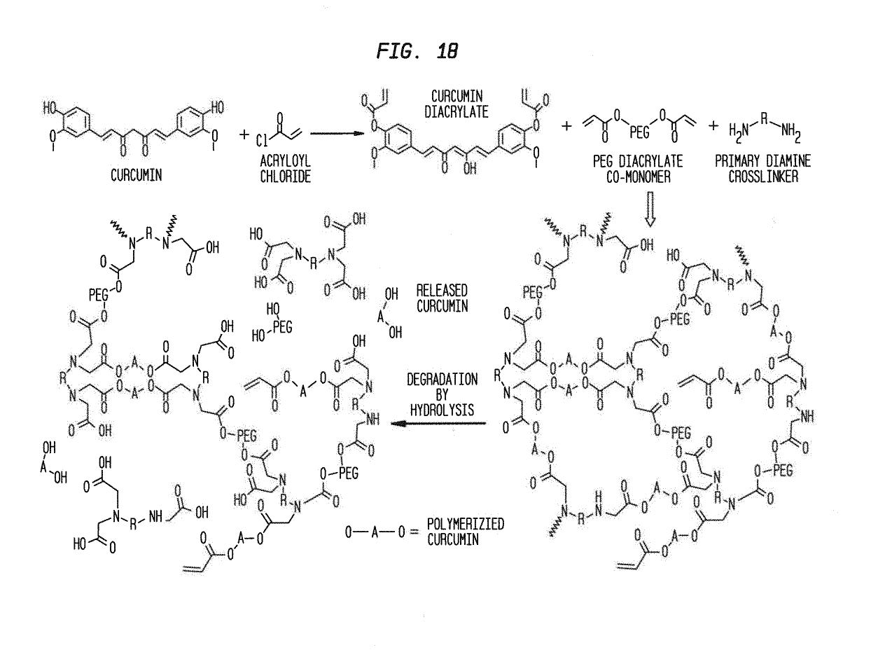

Poly(TAA), is in the form of an intra-articular injection composed of sustained-low dose-releasing polymer microparticles of TAA. TAA is usefully converted into biodegradable crosslinked poly(beta-amino ester) (PBAE) polymers (Poly(TAA)). To do this, TAA is first converted into TAA diacrylate via its hydroxyl groups (FIG. 18). TAA diacrylate is reacted with a diacrylate co-monomer (e.g. poly(ethylene glycol) diacrylate (PEGDA)) and a di-primary amine crosslinker to yield crosslinked Poly(TAA) films (FIG. 18). These films are cryogenically ground into microparticles. The microparticle size distribution is kept between 20 μm and 100 μm in order to minimize any inflammatory response and phagocytosis as reported in previous studies. This size range is also within that reported for triamcinolone acetonide crystalline suspensions marketed as intra-articular injections (e.g. Kenalog).

A useful embodiment of the present disclosure is a two-component system: 1) a sterile septum-top vial containing a single dose of desiccated Poly(TAA) microparticle powder and, 2) a vial containing the liquid injection dispersant. After transferring the dispersant into the Poly(TAA) vial using a sterile syringe, the microparticles are dispersed thoroughly. The suspension is immediately injected into the joint synovial fluid. The original TAA is released continuously for 4 weeks in the joint by hydrolytic degradation of the polymer.

### Example 12

### Treatment of Chronic Wounds

Chronic wounds require a multi-faceted management approach involving, but not limited to, daily inspections, debridement, infection control, moist bandages, compression, pressure offloading, and dietary changes. In cases with compromised blood supply (ischemia), surgical bypass and angioplasty may be required. Hyperbaric oxygen therapy (HBOT) has gained much attention lately with the understanding that increased oxygenation stimulates neovascularization and fibroblast replication, and increase phagocytosis and leukocyte-mediated killing of bacterial pathogens in the wound. However, benefits of HBOT remain controversial, particularly because HBOT itself increases oxidative stress. Advanced therapeutics like human skin equivalents (HSE), e.g., Apligraf and Dermagraft promote healing via the action of cytokines and dermal matrix components. While HSEs have proven to be beneficial, studies have reported that as high as 60% of patients fail to respond. HSEs also suffer from contraindication for already infected/necrotic wounds, high cost, supply-demand challenges, short shelf life, immune rejection, infection transmission, and limited size and shape. Numerous growth factors alone have also been evaluated, but few have progressed to a clinically useful therapy, and all of them suffer from limitations similar to the HSEs. In addition, Regranex (human platelet-derived growth factor), the only FDA-approved growth factor for wound healing, has been issued a black box warning (predisposition of patients to systemic neoplasia). Unfortunately, despite simultaneous use of multiple interventions, most chronic wounds take months to heal or do not heal at all.

This significantly unmet need is addressed by simultaneously targeting two mechanisms involved in perpetuating and mitigating chronic wounds. First, strong evidence has implicated oxidative stress, i.e. excessive production of reactive oxygen species (ROS) as a key pathway in perpetuating the inflammatory phase in chronic wounds. By damaging cellular proteins, membrane lipids, and DNA, oxidative stress impairs cellular processes, such proliferation, migration, and extracellular matrix deposition, critical for wound healing. Wounds of healing-impaired diabetic mice were shown to have reduced levels of the endogenous antioxidant glutathione (GSH) compared to non-diabetic mice. Curcumin, a potent antioxidant, has shown efficacy in accelerating wound healing in preclinical models by controlling both 1) oxidative stress (by reducing lipid peroxidation) and, 2) inflammation (by increasing cell migration and proliferation, increasing expression of transforming growth factor-β1 (TGF-β), fibronectin and collagen, and decreasing production of matrix metalloproteinase). Although all the aforementioned studies achieved accelerated wound healing with curcumin treatment, unless a controlled release formulation was employed as in one of these studies, daily dosing of free curcumin was necessary. This is attributed to known problems with curcumin's bioavailability in vivo, stemming from its short physiological half-life and poor aqueous solubility.

Second, resolvins were recently discovered to be produced endogenously in humans to terminate and resolve the inflammatory phase in a number of pathologies, including wound healing. Resolvins act by reducing neutrophil infiltration, decreasing pro-inflammatory mediators and promoting macrophage phagocytosis of apoptotic cells and microbes. Considering the fact that prolonged, non-resolving inflammation is the hallmark of chronic wounds, we believe it is logical to evaluate resolvins as a novel treatment to promote healing. Further support comes from a recent study wherein attenuated resolvin synthesis was detected in diabetic wounds. Moreover, local application of resolvin D1 (RvD1) accelerated wound healing in diabetic mice by blunting systemic inflammation and restoring macrophage phagocytosis of apoptotic cells.

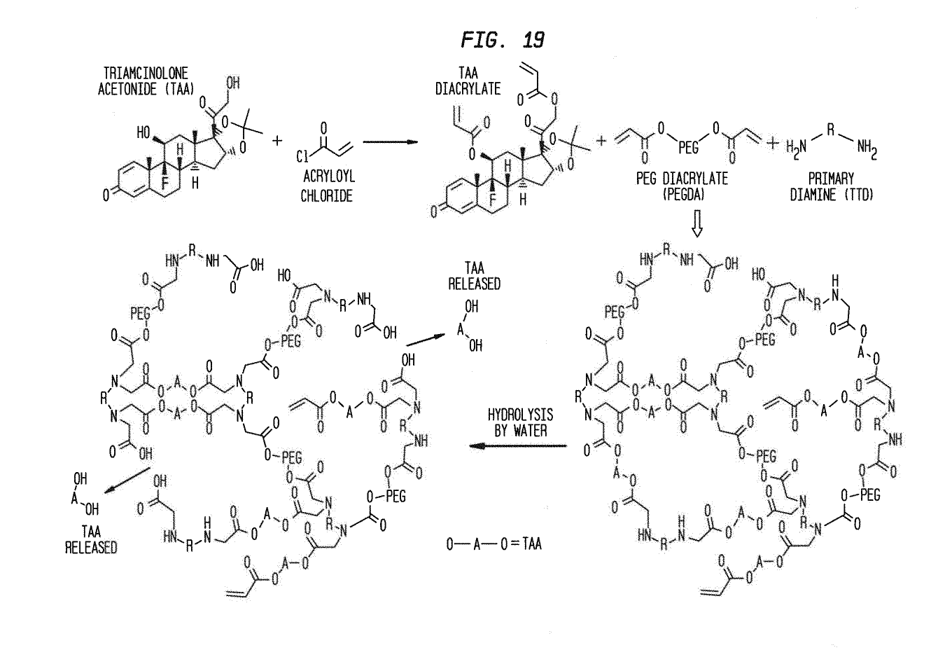

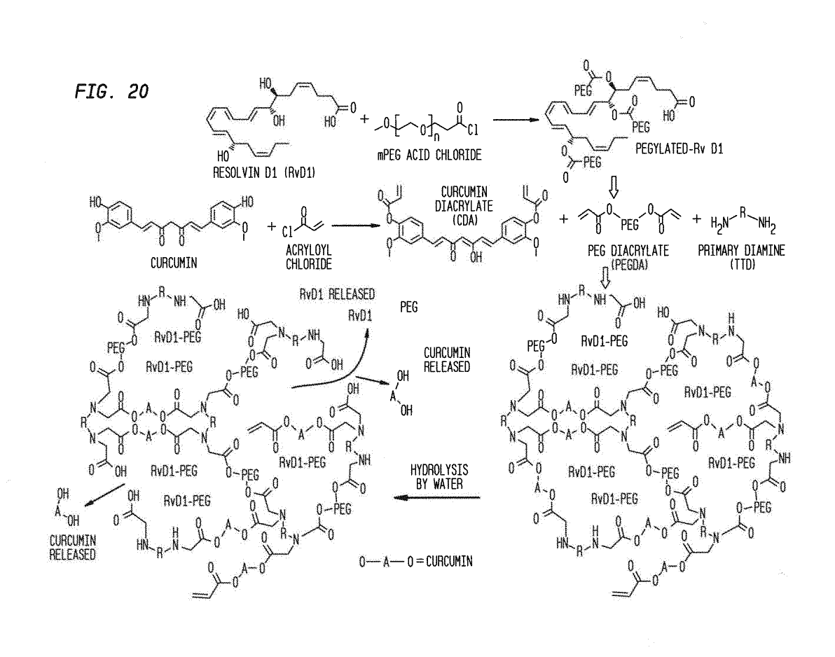

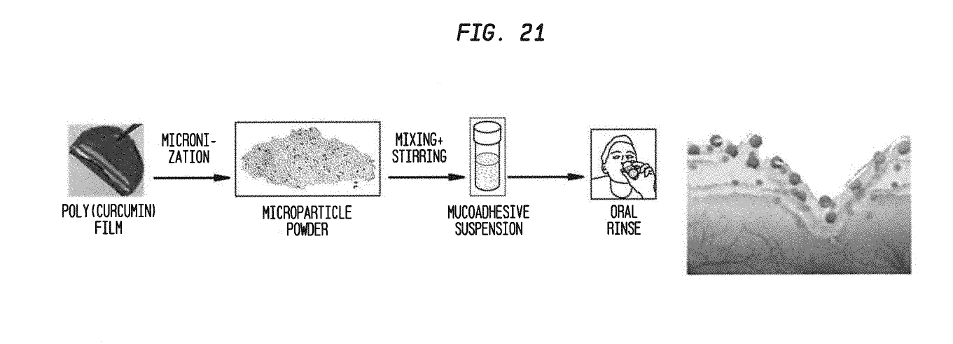

One useful embodiment of the disclosed material is a dual-action topical formulation that utilizes both the aforementioned agents to promote healing. The product, RvD1-Poly(Curcumin), contains a sustained-release polymer of curcumin that simultaneously releases RvD1. The target duration of sustained-release for both curcumin and RvD1 is 3 days, which reduces the reapplication frequency, and therefore provide the option of lowering the dressing change frequency. Curcumin is converted into a biodegradable crosslinked curcumin-poly(beta amino ester) (PBAE) polymer, Poly(Curcumin). To do this, curcumin is first converted into CMA via its hydroxyl groups (FIG. 20). Then, CMA is reacted with a diacrylate co-monomer (e.g. poly(ethylene glycol) diacrylate) and a di-primary amine crosslinked to yield crosslinked Poly(Curcumin) films (FIG. 20). In order to prolong the diffusion-triggered release of RvD1 to 3 days, the hydrodynamic volume of RvD1 is increased by linking poly(ethylene glycol) (PEG) chains to the hydroxyl groups of RvD1 via water-cleavable ester bonds (FIG. 19). The PEGylated-RvD1 (RvD1-PEG) is added during the polymerization reaction to incorporate it into the Poly(Curcumin) matrix (FIG. 20). These RvD1-Poly(Curcumin) films are then cryogenically ground into microparticles and blended with dry PEG as a water-soluble excipient.

A useful product delivery form is a two component system: 1) a sachet containing a single dose of desiccated RvD1-Poly(Curcumin) powder blended with PEG, and 2) a vial containing a single dose of saline. The healthcare professional gently mixes the two components just before administration to form an easy-to-spread ointment. Slow hydrolytic degradation releases curcumin along with RvD1 (FIG. 20).

### Example 13

### Treatment of Oral Mucositis

Oral Mucositis (OM) is one of the most common side effects seen in patients receiving anti-cancer chemo- and radiation-therapy. OM manifests as erythema, which leads to large, contiguous ulcers that can cover >50% of the oral surface. The unbearable pain and hindered oral function, including reduced nutritional intake and dysphasia, can force patients to halt their life-saving anti-cancer therapies. The current standard of care for OM is primarily palliative, typically involving intravenous analgesics, and hourly analgesic and lubricating oral rinses. Unfortunately, their general lack of effectiveness and high treatment burden pose significant patient compliance issues for clinicians. The repeated failure of other products to treat OM further highlights the clear unmet need for effective and safe therapeutic agents and treatment strategies.

Studies have clearly linked OM development to oxidative stress, i.e. the excessive production of reactive oxygen and nitrogen species (ROS & RNS) due to the anti-cancer chemo- and radiation-therapies. Oxidation of proteins, lipids and DNA damages the rapidly-replicating oral mucosal tissue. Oxidative stress also activates key mediators of pro-inflammatory pathways, such as nuclear factor-kappa B (NF-κB), activator protein-1 (AP-1), and STAT3, leading to the further release of tissue-damaging cytokines and ROS, propagating the cycle of cell death and tissue damage. The apoptotic and necrotic chain of events halt epithelial proliferation, ultimately resulting in ulceration.

A multi-action compound with antioxidant and anti-inflammatory properties is an effective treatment strategy. Curcumin, from the Indian spice turmeric, was recently shown to significantly reduce the incidence and severity of chemotherapy- and radiation-induced OM in rats. Curcumin in fact inhibited NF-κB, which in turn decreased inflammatory cytokine production and caspase-induced apoptosis. In response, the Mucositis Study Group of the Multinational Association of Supportive Care (MASCC), the organization that sets clinical guidelines for OM management, recommended curcumin as a new agent of interest.

A recent human clinical trial showed a significantly delayed onset and reduced severity of OM in patients that used turmeric oral rinses compared to povidone-iodine rinses. Although this trial used curcumin's source, turmeric, other studies have linked the bioactivity of turmeric primarily to curcuminoids, particularly curcumin. A point to note is the high dosing frequency that was necessary to achieve this outcome: 4 oral rinses 6 times a day. This is likely related to the reported 2.42% curcumin content in turmeric, along with the known poor bioavailability of curcumin, which is in turn due to a combination of rapid degradation, first-pass metabolism and poor aqueous solubility. Delivery of active curcumin to the disease site via reasonable doses has remained a major barrier to clinical success. Another recent clinical trial in pediatric patients receiving doxorubicin chemotherapy showed that twice daily oral rinsing with a curcumin suspension significantly reduced OM severity compared to historical clinical studies.

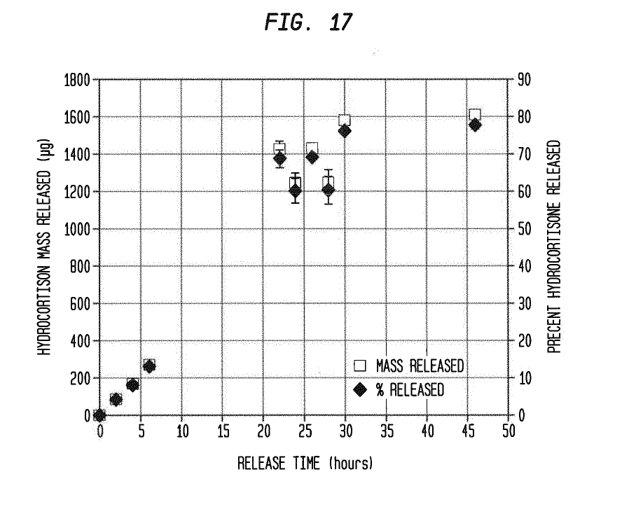

Prolonged and local delivery of curcumin to buccal tissues increases efficacy in treating OM and improves patient compliance. This is achieved by using curcumin converted into biodegradable curcumin-poly(beta-amino ester) (PBAE) polymer (poly(curcumin)). This involves the conversion of a polyphenolic compound, like curcumin, into an acrylate ester (FIG. 18). The phenol acrylate (CDA in this case) is reacted with a primary diamine crosslinker, like 4,7,10-Trioxa-1,13-tridecanediamine (TTD), via the Michael addition to yield crosslinked poly(curcumin) (FIG. 18). The hydrophilicity of the polymer, and thus its degradation rate, is controlled by including a diacrylate co-monomer like poly(ethylene glycol) diacrylate (PEGDA) in desired proportions during the reaction (FIG. 18). The ratio of total acrylate groups to amine protons (RTAAP) can be altered to control the surface chemistry. For example, an RTAAP>1.0 provides excess acrylate groups while an RTAAP<1.0 provides excess amine groups. Upon in vivo delivery, water slowly breaks the ester bonds located between the curcumin and amine groups, releasing the original curcumin molecule (FIG. 17) in a sustained fashion. PEG and acid-modified amine are released as byproducts. Both curcumin and PEG are General Recognized As Safe (GRAS) by the FDA.

Curcumin is structurally protected from premature degradation compared to encapsulation/complexation technologies; and high curcumin loadings, exceeding 40 wt % of the polymer is obtained. This is compared to a maximum of 20% with existing technologies. Aldo tunable sustained release durations ranging from 2 hrs to multiple days are possible. Curcumin is released upon hydrolysis of the polymer; and it is possible to avoid potentially toxic additives (e.g., initiators, plasticizers, surfactants).

In one embodiment a poly(curcumin) oral rinse is a drug-device combination product (FIG. 21) comprised of (1) a sachet containing a single dose of poly(curcumin) microparticles, and (2) a sealed plastic bottle containing 5 ml of mucoadhesive solution. The poly(curcumin) average microparticle size is maintained at 5 μm to eliminate patient discomfort from an unpleasant gritty texture. The mucoadhesive solution is an aqueous solution of pharmaceutical-grade pH-sensitive polymers of acrylic and methacrylic acid (all approved by the FDA for use as inactive excipients). Flavoring agents may also be added to mask any unpleasant taste. The dosage frequency is 1-3 times daily, or as prescribed by the physician. The product is easily used by the patient with minimal training at home or in clinics. Typically, sachet contents are added to the bottle and thoroughly mixed with the mucoadhesive solution to form a suspension (FIG. 21). The patient swishes the suspension in the mouth for 1-2 minutes, and expectorate any excess.

Degradation of the poly(curcumin) can release EDTA-like multi-acid by-products, as exemplified in FIG. 18. Any potential harmful effects of these by-products, can be counteracted by inclusion of neutralizing agents, like calcium-based salts, either in the ground microparticles or in the mucoadhesive solution, or both.

Noted in the use of this composition are physical barrier protection along with curcumin delivery to control oxidative stress and continuous release of amounts of curcumin, instead of a single burst. This alleviates solubility limitations and improves absorption into tissue. Noted too are high loading and sustained release of curcumin for up to 24 hours reduces treatment dose and frequency. Further noted are localized delivery to the buccal tissues reduces risk of systemic side effects. Delivery as a mucoadhesive suspension offers complete coverage of the complex oral cavity as well as easy use by patients: mix→rinse in mouth→expectorate.

### Example 14

### Improved Management of Oral Mucositis

As described above, at present, the management of OM primary involves palliative treatment with mucoadhesive oral rinses that deposit a protective polymer-based barrier film on the buccal tissues. These barrier products reduce pain and improve comfort by preventing mechanical injury and maintaining tissue hydration. At present, patients reapply such oral rinses multiple times a day as and when they feel necessary based on reduced effectiveness in the mouth. It will be greatly beneficial if these barrier rinses contained a visual indicator that aids patients in determining a suitable time to reapply the oral rinse.

A product, similar to the only described in Example 12 serves this purpose. The poly(curcumin) microparticles have an intense orange color (FIG. 21) that serves as an indicator of the presence or absence of the barrier on the buccal tissues. As the barrier erodes, so will the poly(curcumin) microparticles, causing fading of the orange color. The patients can reapply the poly(curcumin) oral rinse when the color reaches a certain level or disappears completely, based on their preference or as directed by the physician.

### Example 15

### Improved Antioxidant Delivery for Cosmetics

Skin is exposed to damage resulting from various sources, including both environmental factors and biochemical processes. Oxidative processes damage proteins, lipids, and other cellular components necessary to maintain the health and appearance of skin, resulting in skin changes, such as skin aging (e.g., age spots), hyperpigmentation, UV damage, lines, wrinkles, uneven skin texture (e.g., cellulitis), etc. Oxidative damage to the skin and its more detailed causes are listed in Miyachi, Y: “Skin diseases associated with oxidative injury,” Fuchs J, Packer L (eds.), Oxidative Stress In Dermatology, Marcel Dekker, New York, pp. 323-331 (1993).

The damaging effects of the UV part of solar radiation on the skin are generally known. While rays having a wavelength which is less than 290 nm (the UVC range), are absorbed by the ozone layer in the earth's atmosphere, rays in the range between 290 nm and 320 nm (the UVB range), cause an erythema, simple sunburn or even more or less severe burns. The narrower range around 308 nm is given as a maximum for erythema activity of sunlight. Further, UV radiation is ionizing radiation. Hence, there is the risk that ionic species are produced on UV exposure, which then in turn are able to intervene oxidatively in the biochemical processes.

UV radiation, however, may also lead to photochemical reactions, wherein then the photochemical reaction products intervene in the skin mechanism. Predominantly such photochemical reaction products are free radical compounds, for example hydroxyl radicals. Also, undefined free radical photoproducts, which are produced in the skin itself, may trigger uncontrolled side reactions due to their high reactivity. Singlet oxygen, a non-free radical excited state of the oxygen molecule, however, may occur in UV irradiation, short-lived epoxides and many others. Singlet oxygen, for example, is characterized with respect to the normally existing triplet oxygen (free radical base state) by increased reactivity. Nevertheless, excited, reactive (free radical) triplet states of the oxygen molecule also exist. Furthermore, there is the occurrence of lipid peroxidation products, such as hydroperoxides and aldehydes, wherein first in turn free radical chain reactions may be triggered and to which overall cytotoxic properties have to be ascribed (Michiels and Ramacle, Toxicology, 66, 225 ff. (1990)). Lipid peroxidation is an oxidative process that degrades lipids, wherein free radicals steal electrons from the lipids in cell membranes, causing oxidative stress and cell damage.

In order to prevent these reactions, additional antioxidants and/or free radical absorbers/scavengers may be incorporated in cosmetic or dermatological formulations. Antioxidants are substances that scavenge free radicals and prevent oxidation processes or prevent the auto-oxidation of fats containing unsaturated compounds. Antioxidants are mainly used as protective substances against the decay of the compositions containing them. However, it is known that undesirable oxidation processes may also occur in the human and animal skin. Such processes play a considerable part in skin aging. Thus, antioxidants and/or free radical absorbers may additionally be incorporated into cosmetic formulations to treat or prevent damage caused by oxidative and degenerative biochemical processes.

Antioxidant molecules used in such applications to protect skin from damage and premature ageing include, but are not limited to, vitamin E, curcumin, resveratrol, quercetin, and ascorbic acid. More broadly they include, but are not limited to, curcuminoids, stilbenoids, phenylethanoids, tocopherols, tocotrienols, flavanones, flavones, prenylflavonoids, isoflavones, isoflavanes, dihydrochalcones, isoflavenes, coumestans, lignans, flavonoligans, flavonols, tannins, catechols, catechins, and cannabinoids.

One of the technical difficulties for the use of the above antioxidant compounds is their instability. In particular in cosmetic formulations possessing a protective activity against the damages produced by exogenous factors, such as oxidant agents, pollutants and UV radiations. For example, the strong chemical activation energy derived from ultraviolet radiation induces hydrolytic reactions, with a consequent reduction or loss of the antioxidant, filtering and protecting activities. A reduction of UVB filtering capacity leads to a reduction in SPF of the formulation, and therefore to a higher burn risk, while a reduced capacity to filter UVA radiation might go unnoticed, exposing to a greater risk of adverse chronic effects, which are characteristic of these bands. Furthermore, the photo-degradation of the antioxidant molecule generates potentially harmful chemical substructures which induce allergic sensitization processes, skin irritation or toxicity phenomena due to their trans-dermal absorption. For example, it is well-known that trans-resveratrol is unstable in solution when exposed to light and readily isomerizes to the cis isomer. Additionally and without being bound by any particular theory, it is believed that resveratrol may also undergo an auto-oxidation process, especially when in solution, which leads to the production of O₂^{•-}, H₂O₂, and a complex mixture of semiquinones and quinines that may be cytotoxic. These auto-oxidation or degradation events are important, because oxidized resveratrol generates complexes with others molecules, such as copper ions. The oxidative product of resveratrol is a dimer, and the initial electron transfer generates the reduction of Cu(II) to Cu(I). Thus, the copper-peroxide complex is able to bind DNA and to form a DNA-resveratrol-Cu(II) ternary complex. These complexes favor and give rise to internucleosomal DNA fragmentation, which is a hallmark of cell death. Such transformation diminishes its physiological properties, mainly under use conditions where the compound is exposed to the atmospheric air, metallic ions and water such as, for example, when incorporated into a topical cosmetic. The poly(resveratrol) microparticles provide protection from auto-oxidative conversion of resveratrol in cosmetic products.

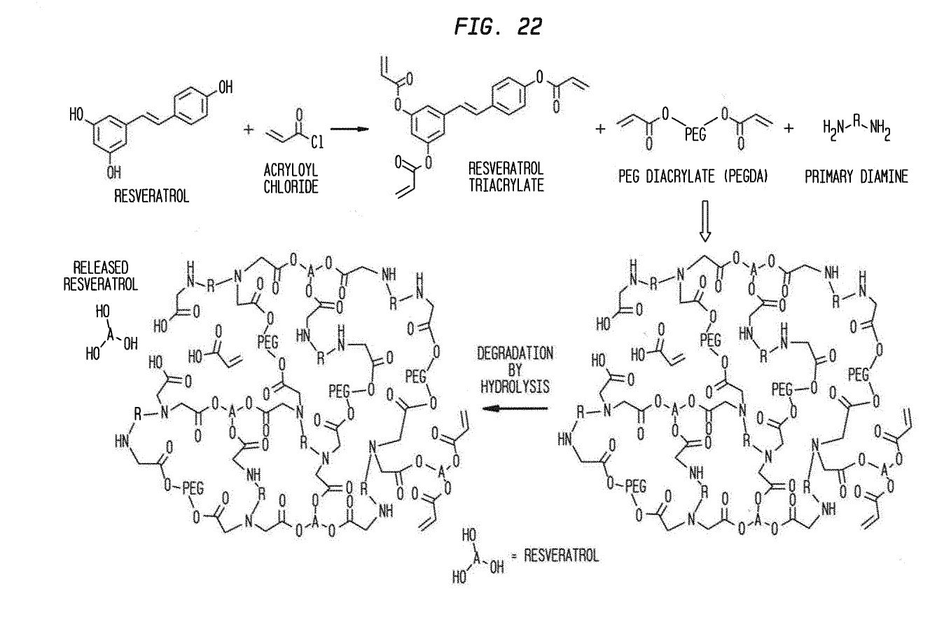

In one embodiment the disclosed product is composed of an of an antioxidant, like resveratrol, converted into microparticles of biodegradable poly(beta-amino ester) (PBAE) polymers. Specifically, the polyphenolic antioxidant, like resveratrol, is first converted into an acrylate ester (FIG. 22). The phenol acrylate (e.g. resveratrol triacrylate (RTA)) is then reacted with a primary diamine crosslinker, like 4,7,10-Trioxa-1,13-tridecanediamine (TTD), via the Michael addition to yield crosslinked poly(resveratrol) (FIG. 22). The hydrophilicity of the polymer, and thus its degradation rate, is controlled by including a diacrylate co-monomer like poly(ethylene glycol) diacrylate (PEGDA) in desired proportions during the reaction (FIG. 22). Upon application to the body, water slowly breaks the ester bonds located between the resveratrol and amine groups, thereby releasing the original curcumin molecule (FIG. 22) in a sustained fashion.

Note that resveratrol is believed structurally protected from premature degradation compared to encapsulation/complexation technologies. It further offers high resveratrol loadings, exceeding 20 wt % of the polymer versus a maximum of 20% with existing technologies. Also note the advantage of tunable sustained release durations ranging from 2 hours to days. Original, active, resveratrol is released upon hydrolysis of the polymer by the disclosed composition and method with no requirement for additives (e.g., initiators, plasticizers, surfactants).

1. A polymerized hydro-X compound.

2. The polymerized hydro-X compound of claim 1 where the hydro-X compound is selected from the group consisting of curcuminoids, stilbenoids, resolvins, phenylethanoids, tocopherols, tocotrienols, flavanones, flavones, prenylflavonoids, isoflavones, isoflavanes, dihydrochalcones, isoflavenes, coumestans, lignans, flavonoligans, flavonols, mycoestrogens, xenoestrogens, phytoestrogens, sterols, corticosteroids, androgens, estrogens, stanols, steroids, secosteroids, tannins, statins, catechols, catechins, opioids, cannabinoids, pleuromutilins, luteolinidin, anthocyanidins, apigeninidin, glycosylated compounds, or macrolides.

3. The polymer of claim 2 wherein said particle is in the form of a film.

4. The polymer of claim 2 wherein said polymer is in the form of microparticles.

5. The microparticles of claim 4 wherein at least about 90% of said particles are less than about 10 μm in diameter and about 50% are less than about 5 μm in diameter.

6. The microparticles of claim 5 wherein at least about 90% of said particles are less than about 3 μm in diameter and about 50% are less than about 1 μm in diameter.

7. The film of claim 3 where the hydro-X compound is released from said polymer in a controlled steady state fashion with substantial release at from about 12 hr to about 4 weeks.

8. The microparticles in claim 4 where the hydro-X compound is released from said polymer in a controlled steady state fashion with with substantial release at from about 12 hr to about 4 weeks. A polymerized hydro-X compound in the form of a mucoadhesive suspension.

9. The mucoadhesive suspension in claim 9 where the hydro-X compound is curcumin or resveratrol.

10. The mucoadhesive suspension of claim 10 wherein the hydro-x compound is resveratrol.

11. The polymerized resveratol of claim 11 in the form of microparticles.

12. The microparticles of claim 12 wherein at least about 90% of said microparticles are less than about 3 μm in diameter and about 50% are less than about 1 μm in diameter.

13. A method to treating a patient for osteoartritis by the step of administering a therapeutically effective dose of a compound of claim 1.

14. A method to treating a patient for oral mucositis by the step of administering a therapeutically effective dose of a compound of claim 1.

15. A method of claim 14 wherein said compound is in the form of a mucoadhesive solution of poly(curcumin) or poly(resveratrol) microparticles.

16. A method to treating a chronic wound in a patient by the step of administering therapeutically effective dose of a compound of claim 1.
